(19) United States
(12) Patent Application Publication
(10) Pub. No.: US 2013/0052636 A1
(43) Pub. Date: Feb. 28, 2013

(54) RAPID DETECTION OF METABOLIC ACTIVITY

(71) Applicants: Ravi Kant Verma, La Canada, CA (US); Kenneth Mathew Zangwill, South Pasadena, CA (US); Ashraf Samir Ibrahim, Irvine, CA (US)

(72) Inventors: Ravi Kant Verma, La Canada, CA (US); Kenneth Mathew Zangwill, South Pasadena, CA (US); Ashraf Samir Ibrahim, Irvine, CA (US)

(73) Assignee: SPECTRAL PLATFORMS, INC., La Canada, CA (US)

(21) Appl. No.: 13/590,915

(22) Filed: Aug. 21, 2012

Related U.S. Application Data
(60) Provisional application No. 61/526,160, filed on Aug. 22, 2011.

(51) Int. Cl. G01N 21/65; C12M 1/42

(52) U.S. Cl. 435/5; 435/34; 435/32; 435/288.7

(57) ABSTRACT

Some aspects of the invention provide for a method for detecting metabolic activity in a sample by obtaining a sample, illuminating the sample at a plurality of time points, measuring transmitted light from a marker of metabolic activity in the sample at the plurality of time points, and detecting the presence or absence of metabolic activity from a change in the transmitted light at the plurality of time points. Other aspects of the invention provide for a method for detecting metabolic activity in a sample by providing a sample have a detectable marker therein that is reflective of metabolic activity in the sample, producing an amplified signal from the marker, measuring the amplified signal at a plurality of time points, and detecting metabolic activity from a change in the signal. Additional aspects provide for a system for detecting metabolic activity in a sample.

### CROSS-REFERENCE TO RELATED APPLICATIONS

This application claims the benefit of priority to U.S. Provisional Application No. 61/526,160, filed Aug. 22, 2011; which is hereby expressly incorporated by reference in its entirety.

### STATEMENT REGARDING FEDERALLY SPONSORED R&D

Portions of this invention may have been made with United States Government support. As such, the United States Government may have certain rights in the invention.

### BACKGROUND

1. Field of the Invention

Methods and systems for the detection of metabolic activity in a sample, particularly the detection and/or characterization of pathogens and toxins in samples by detecting metabolic activity.

2. Description of the Related Art

Patients who display symptoms of an infection (bacterial, viral or fungal) are often given antimicrobial drugs. Made more important by the recently increased development of drug-resistant bacterial pathogens, some currently untreatable, it is important to correctly identify the pathogen, and to prescribe a drug regimen that is correctly tailored to the pathogen. However, the identification of pathogens that cause infectious diseases takes a long period of time, usually 48-72 hours using present techniques. This delay often requires administration of broad-spectrum empiric antimicrobial therapy that is necessarily not guided by specific information from the laboratory with regard to identification of the pathogen or its susceptibility to specific therapies. Such uninformed empiric therapy may lead to important secondary complications such as drug reactions, development of antimicrobial resistance, and performance of unnecessary diagnostic testing. It is these considerations that have driven a large effort to develop tools for the more rapid diagnosis and characterization of such infections.

Amongst established methods for rapid diagnosis, polymerase chain reaction (“PCR”)-based methods (which detect nucleic acid material) are the most developed as a potential rapid diagnostic tool. This method, however, is pathogen-specific and requires that a molecular target for the pathogen be first identified and developed. Additionally, these PCR-based methods do not provide any information on the pathogen's antimicrobial susceptibility unless that information has been previously developed. There are several other documented methods for developing information on susceptibility (or resistance) of a pathogen to a particular drug. These methods, however, require pre-growth from a previously obtained clinical sample and therefore do not provide information in a timely manner.

For instance, U.S. Pat. No. 3,772,154 describes a method and apparatus for automated antibiotic susceptibility analysis of bacterial samples wherein the clinical sample is divided into several aliquots and fed into one or more receptacles containing various antibiotics and wherein the aliquots are incubated for several hours, and then killed and the bacteria count determined by various means. Since the method relies on counting bacteria cells, a large number of cells must be present before accurate susceptibility information can be developed. This requirement makes this method impractical for clinical samples, especially when low levels of pathogen loadings are encountered.

U.S. Pat. No. 3,983,006 describes a method for determining minimum inhibitory concentration (“MIC”) of an antibiotic by continuously measuring the change in optical properties in response to the bacterial growth rate of a bacterial suspension in the absence and the presence of the antibiotic. U.S. Pat. No. 4,132,599 describes a method for antimicrobial susceptibility determination for bacteria in infected urine samples without any isolation of the bacteria. Their method is based on a bacterial ATP assay and requires the elimination of non-bacterial ATP.

U.S. Pat. No. 4,146,433 describes a method whereby bacteriostatic activity can be obtained by an Agar dilution method. Agar plates are prepared that contain dilutions of antibiotics, test bacteria are inoculated into the agar plates, and MIC values are obtained. Subsequently, an antibiotic inactivating enzyme solution is sprayed onto the plates to inactivate the antibiotic. After further incubation, the minimal concentration at which no visible growth occurred on the plates is determined and is defined as minimal bactericidal concentration (“MBC”). This method is the standard by which MIC and MBC values are obtained today. However, this method generally takes over 24 hours to provide relevant information.

U.S. Pat. No. 4,209,586 describes a method whereby changes in the redox potentials of cultures of a microorganism with and without a tested growth inhibiting agent are monitored during the phase of growth in which the redox potential is normally positive and the rate of potential change is approximately linear. Effective growth inhibiting agents produce a measurable decrease in the change of the redox potential to a more negative value within less than one hour. A sensible signal indicative of the growth inhibiting action of the tested agent may be obtained from a comparator by storing an amplified signal indicative of the redox potential at a first time and feeding the stored signal together with another amplified signal obtained less than one hour thereafter to the comparator.

U.S. Pat. No. 4,236,211 describe a method that develops a fixed functional relationship in either tabular or equation form between the growth of various microorganisms in the presence of a few (e.g., one or two) concentrations of a predetermined antibiotic and the minimum concentration of such antibiotic necessary to at least inhibit the activity of such samples. The relationships are established for each desired combination of antibiotic and general class of microorganisms and the degree of growth is measured at a predetermined time or level of growth, preferably before saturation occurs at “growth” or “no growth” extremes. The minimum concentrations used in deriving these fixed relationships are determined by standard accepted quantitative techniques. Thereafter, the minimum concentration of the predetermined antibiotic necessary to at least inhibit the activity of any given pathogenic microorganism taken from the same predetermined general class of organisms may be rapidly and accurately determined by (1) measuring the growth of such sampled pathogenic organism after the same predetermined time in the presence of the same few (e.g., one or two) predetermined concentrations of the antibiotic and (2) using the resulting measurements together with the previously established fixed functional relationship to identify the required minimum concentration for that particular combination of microorganism and antibiotic. An apparatus for semiautomatically and for automatically carrying out this method are also disclosed.

U.S. Pat. No. 4,252,897 describe a method and apparatus for bacterial testing, in which a multiple-pin inoculation head picks up bacterial samples in a compartmentalized sample tray and transfers these to a compartmentalized culture plate for incubation. The culture plate contains a test medium, presumably and antibiotic, to which some bacteria are sensitive. An indicator material is also included, which changes color upon pH change due to bacterial growth. The result is a geometric pattern of color, which is entered into computer storage. Additional culture plates are also inoculated in the same way, each containing a different test medium together with an indicator. After all plates have been incubated and data entered into the storage, a computer facility compares the pattern of sensitivity of the unknown bacteria with known sensitivity patterns of known bacteria to determine the most likely identification for each of the unknown bacterial samples.

U.S. Pat. No. 4,132,599 describes a method for determining bacterial sensitivity, wherein aliquots of bacterial suspension in a culture medium, the antimicrobial drug, and tritiated thymidine are deposited into polyethylene tubes having two compartments separated by a fine filter. A precipitation agent (such as trichloroacetic acid) is deposited into the polyethylene tubes to precipitate the bacteria. Following this, a vacuum system is used to draw out the liquid through the filters, and the filtered bacteria are counted. As with U.S. 3,772,154, this method relies on counting of bacteria cells, which requires that a large number of cells be present.

U.S. Pat. No. 4,448,534 describes an apparatus for antibiotic susceptibility testing wherein the bacteria count in a multi aliquot tray is determined by optical density methods. This method also requires the presence of a large number of bacteria cells. U.S. Pat. No. 4,604,351 describes a similar device, but wherein the uptake of a labeled nucleotide is used to determine bacterial growth in the presence of various chemical agents. This method is limited to bacterial cells that will uptake labeled nucleotides.

U.S. Pat. No. 6,750,038 describes a device wherein the susceptibility of bacteria to antimicrobial that are known to inhibit specific enzymatic pathways within the bacteria are measured. In this case, the resistance is characterized by the suppression or expression of the particular enzyme that corresponds to the resistance mechanism. This method is limited to bacteria with specific resistance mechanisms only. U.S. Pat. No. 6,861,230 describe an assay for adenylate kinase in an in vitro test for the external conditions of growth for bacteria cells.

U.S. Pat. No. 7,081,353 describes a device for drug susceptibility testing that is based on the rate of oxygen consumption in samples with varying bacteria and antimicrobial concentrations. Measuring drug susceptibility by detecting dissolved oxygen concentration with an oxygen electrode is well known, but typically takes a long time (several days). Machida et al. describe improvements whereby this time period can be shortened to a few hours. But even so, oxygen measurements are compromised by oxygen consumption due to the metabolism of host cells present in a clinical sample. Thus, none of these methods can be applied to develop susceptibility information directly from clinical samples in a timely manner.

The difficulty of developing pathogen identification and susceptibility information directly in clinical samples can be illustrated with Raman scattering methods. When starting with clinical samples, conventional Raman methods require large sample preparation times (ca. 24 hours) for pathogen identification, and an additional few hours for susceptibility testing. The Raman method involves illumination of the sample of interest (in this case, a sample vial containing the clinical sample with the suspected pathogen). Some of the incident light is incoherently scattered at wavelengths other than the wavelength of the incident light, and a spectrum of the scattered light intensity versus wavelength becomes a signature of the pathogen.

However, the problem arises in the low concentration of pathogens that can be clinically relevant. For instance, a single colony forming unit per milliliter (CFU/mL) of blood would be consistent with a “bacteremia/fungemia” condition in a human being. More typical numbers are 10-100 CFU/mL, and in rare cases the numbers can be as high as 100,000-1,000,000 CFU/mL. Even at 1,000,000 CFU/mL, the clinical sample is dominated by other constituents. For instance, in whole blood, the red blood cells and the white blood cells would both outnumber the bacterial cells. Even if the red blood cells and white blood cells were removed from the blood (e.g., by centrifugation), the pathogens would be dominated by the intrinsic serum proteins, whose Raman spectrum is similar to the Raman spectrum of pathogens.

Specifically, at around 10,000,000 CFU/mL, the serum sample would have a pathogen content that was comparable to the content of the intrinsic serum components. At 100 CFU/mL, the serum sample has a pathogen content that is 100,000 times less than the intrinsic serum component.

Thus, the Raman spectrum of the clinical sample is dominated by the intrinsic component of the clinical sample. This is the underlying reason why previous methods require that the pathogen must be isolated, and cultured, before a useful Raman spectrum can be acquired from it. This difficulty with the implementation of the Raman method is also applicable to other methods.

Despite these limitations, several Raman-based methods have been proposed for pathogen identification and susceptibility measurements. In all of these methods, various mechanisms are used to overcome the low clinical concentrations of the pathogen.

For instance, U.S. Pat. No. 5,866,430 describes a method for detecting & identifying chemical and microbial analytes. The method comprises four basic steps, with the first step being a bioconcentrator that attempts to concentrate the microbial cells. U.S. Pat. No. 5,573,927 describes a method whereby the Raman spectra of a first set of target cells of an initially cultured bacteria E. coli is compared with the Raman spectra of the same cells cultured in the presence of antibiotics, and the comparison is used to develop information on the bacteria's resistance to the antibiotic. In this case, the sample consists of E. coli cells that have been isolated and cultured, thus the methods cannot be applied to clinical samples, but the difference spectrum method is used to develop additional sensitivity. U.S. Pat. No. 6,040,906 describes a method whereby Resonance Raman methods are used to identify various organic and inorganic components of biomatter. Previously, U.S. Pat. No. 4,847,198 have shown that resonance Raman spectra of pure cultures of bacteria exhibit taxonomic identifiers. By collecting resonance Raman spectra as a function of laser excitation frequency, these inventors claim a method of taxonomic identification using the excitation behavior of the Raman spectra of the species in question. U.S. Pat. No. 6,379,920 describes a method whereby the Raman spectrum of a clinical sample from a non-infected patient is used as a reference that is subtracted from the Raman spectra of an unknown clinical sample. With this method, the inventors claim that specific bacteria can be identified sooner and without culturing. However, this method does require a significant bacterial cell count, such that the Raman spectrum of the sample with the pathogen is different from the one without.

U.S. Pat. No. 7,256,875 and U.S. Pat. No. 7,262,840 describe methods for the detection and identification of pathogenic microorganisms via Raman imaging. While these methods can be used to detect pathogens in normally clear samples (such as cryptosporidium in municipal water supplies, as described in U.S. Pat. No. 7,428,045), these methods cannot be applied to clinical samples where a large number of cells with a similar Raman signature are expected to be present.

Given these limitations in the current state of the arts, it is desirable to develop a method and system that can rapidly identify and characterize the susceptibility of an unknown pathogen present in a clinical sample to an antimicrobial agent without requiring any isolation steps. We have now discovered methods and systems for the detection of metabolic activity in a sample, particularly the detection and/or characterization of pathogens and related substances in samples by detecting metabolic activity.

### SUMMARY

Some embodiments are a method for detecting metabolic activity in a sample, comprising obtaining a sample; illuminating the sample with substantially monochromatic light at a plurality of time points; measuring Raman scattered light from a chemical marker of metabolic activity in the sample at the plurality of time points; and detecting metabolic activity from a change in the Raman scattered light at the plurality of time points. Other embodiments are a method for detecting metabolic activity in a sample, comprising providing a sample having a detectable marker therein that is reflective of metabolic activity in the sample; producing an amplified signal from the marker; measuring the amplified signal at a plurality of time points; and detecting metabolic activity from a change in the amplified signal at the plurality of time points. Some embodiments are a system for detecting metabolic activity in a sample, comprising a light source; a controller for periodically illuminating a sample with the light source, wherein the sample contains a chemical marker responsive to metabolic activity in the sample; a detector configured to measure a light signal from the marker; and a computer configured to receive light measurements from the detector and ascertain a change in the marker over time that is indicative of metabolic activity in the sample.

In some embodiments, the Raman scattered light is resonance enhanced. In some embodiments, the change in the Raman scattered light is cumulative. In other embodiments, the marker is an anti-oxidant, a free-radical scavenger, a carotenoid, and/or lycopene. In some embodiments, the marker is an element-sequestering protein complex and/or an iron-sequestering protein complex. In some embodiments, the marker increases as a result of metabolic activity. In other embodiments, the marker decreases as a result of metabolic activity.

In some embodiments, the metabolic activity is the production of free radicals, the production of a carotenoid, and/or the sequestering of iron. In some embodiments, the presence of metabolic activity indicates the presence of a pathogen, such as a bacterium, fungus, parasite, or virus. In other embodiments, the metabolic activity indicates that a toxin or other disease-modifying substance released by a pathogen is present in the sample.

In some embodiments, the sample contains an anti-pathogenic substance. The anti-pathogenic substance is optionally configured to allow pathogen identification and/or classification from the detected metabolic activity. In some embodiments, the sample contains a culture broth configured to allow pathogen identification and/or classification from the presence of metabolic activity. The anti-pathogenic substance is optionally selected from the group consisting of an anti-biotic, anti-fungal, and anti-viral substance. Measured metabolic activity indicates effectiveness or ineffectiveness of the anti-pathogenic substance.

In some embodiments, the sample includes a body fluid such as blood, cerebrospinal fluid, and/or urine. The sample is optionally cultured and can include a cultured cell line. In some embodiments, the marker is naturally present in the sample. In other embodiments, marker is added to the sample prior to illuminating. In some embodiments, the sample includes a calibrant Raman marker in the sample or on a sample container. The sample or the sample container are optionally interrogated for the presence or intensity of the calibrant Raman marker.

In some embodiments, the detecting is completed in less than about 6 hours. In other embodiments, the detecting is completed in less than about 30 minutes.

In some embodiments, the light signal from the marker is from Stokes Raman scattering. In other embodiments, the light signal from the marker is from anti-Stokes Raman scattering.

In some embodiments, the amount of the marker accumulates in the sample over time to indicate metabolic activity in the sample. In other embodiments, the marker in the sample decreases over time to indicate metabolic activity in the sample. In some embodiments, the light transmitted by the marker is resonance enhanced.

### BRIEF DESCRIPTION OF THE DRAWINGS

FIG. 1. A flow diagram describing one method of detecting metabolic activity in a sample.

FIG. 2. A flow diagram describing a second method of detecting metabolic activity in a sample.

FIG. 3. A system describing one embodiment for detecting metabolic activity in a sample.

FIG. 4. A schematic describing one embodiment of a method for detecting metabolic activity in a sample using iron sequestering as an indicator of metabolic activity.

FIG. 5. A representative spectrum obtained for one embodiment of a method of detecting metabolic activity in a sample.

FIG. 6. A plot of normalized peak intensity versus time illustrating one method for detecting metabolic activity in a sample.

FIG. 7. A scheme illustrating the sequestering of iron by pathogenic proteins.

FIG. 8. A schematic describing a second embodiment of a method for detecting metabolic activity in a sample using changes in lycopene as an indicator of metabolic activity.

FIG. 9. A table illustrating signal to noise ratios for various detection methods.

FIG. 10. A plot illustrating Iron-Transferrin excitation profiles.

FIG. 11. Raman spectrum demonstrating bacterial classification.

FIG. 12. Data plot illustrating constant peak intensity during a bacterial classification experiment.

FIG. 13. Data plot illustrating a change in peak intensity during a bacterial classification experiment.

FIG. 14. Raman spectrum overlay for a blood sample containing MRSA.

FIG. 15. A plot illustrating the change in peak intensity for various pathogen concentrations.

FIG. 16. A plot illustrating the slope of the biomarker as a function of pathogen concentration.

FIG. 17. A plot illustrating the use of one embodiment to detect the MIC for an antibiotic. This plot illustrates the measurements done to estimate the MIC.

FIG. 18. A plot illustrating the use of one embodiment to detect the MIC for an antibiotic. This plot illustrates the one specific method by which MIC can be estimated.

FIG. 19. A plot depicting the use of a marker to distinguish samples containing multiple pathogens.

FIG. 20. A plot illustrating the use of one embodiment to detect the MIC for samples with multiple pathogens.

FIG. 21. A plot demonstrating the production of lycopene by M. bovis.

FIG. 22. A second plot demonstrating the production of lycopene by M. bovis.

FIG. 23. A plot demonstrating the production of lycopene by M. fortuitum.

FIG. 24. A plot illustrating the detection of a toxin in a sample.

FIG. 25. A second plot illustrating a detectable change over time resulting from the presence of a toxin in a sample.

### DETAILED DESCRIPTION OF THE PREFERRED EMBODIMENT

As used herein, abbreviations are defined as follows:
ATP Adenosine triphosphate
CBC Complete blood count
CFU Colony forming units
cm⁻¹ Inverse centimeters or wavenumber
EDTA Ethylenediaminetetraacetic acid
Fe-Tr Iron-transferrin
MBC Minimal bactericidal concentration
MIC Minimum inhibitory concentration
MDR Multi-drug resistant
nm nanometer
PCR Polymerase chain reaction
apo-Tr Transferrin lacking bound iron
ROS Reactive oxygen species
RNS Reactive nitrogen species
UV Ultraviolet

### Introduction

The need in the art is addressed by a device that monitors changes in metabolic activity in a sample in response to an invading pathogen. The difficulties in the prior art can be ascribed to the general approach of trying to measure the pathogen's concentration or bioburden in the clinical sample. Since the pathogen's concentration is small, compared to intrinsic components in the clinical sample, the measurement is difficult. By contrast, certain disclosed embodiments measure changes in a compound in response to an invading pathogen, including changes that accumulate over time, and including changes from a nominally smaller baseline level. Thus, these embodiments enable characterization of low levels of pathogens present in the clinical samples.

Another embodiment of the invention measures the generation of a compound that can be easily detected in the clinical sample. In all cases (production, consumption, or modification of a marker), the sensitivity of the measurement can be enhanced by appropriate use of a measurement technique that intrinsically amplifies the signal due to the marker.

Some embodiments exploit the use of the iron sequestration process. Every invading pathogen requires iron for growth, and the vertebrate host sequesters iron in special iron containing proteins, such as the iron-transferrin complex present in blood. Accordingly, the pathogen must extract iron from the host proteins. Different pathogens employ various mechanisms to sequester iron from the host protein. If a viable pathogen is present in blood (which happens to be a mostly favorable growth medium for pathogens in all respects, except for the lack of free iron), the iron content of the transferrin is rapidly depleted. By contrast, when viable pathogens are not present in the blood sample, then the iron content of transferrin is maintained.

The presence of iron in the iron-transferrin (Fe-Tr) complex results in intense Raman bands. These Raman bands can be monitored as a marker of the iron content in transferrin with a commercially available Raman spectrometer. For instance, the Fe-Tr band has intense Raman peaks at 1510, 1280, 1160 and 1605 cm⁻¹. The presence of iron in the iron-transferrin (Fe-Tr) complex also results in an optical absorption peak centered at around 470 nm. This optical absorption peak can be monitored as a marker for the iron content in transferrin, with commercially available color sensors.

Thus, one embodiment is a commercially available Raman spectrometer that monitors the 1510 cm⁻¹ Raman peak as a function of time in the clinical sample. The clinical sample requires some processing to transform it into an appropriate growth medium in all respects, except for the minimal availability of free iron, and with the only available iron source being an iron-protein complex. With blood samples, this is accomplished with a centrifugation step or the use of gravity with unclotted blood which separates serum or plasma that contains both the pathogen and the iron-transferrin complex. With urine samples, this can be accomplished by adding a small volume of the urine sample to a larger assay of stock serum. If viable pathogens are present, then the iron content of Fe-Tr will be rapidly depleted. In other embodiments, antimicrobial susceptibility is characterized by repeating the measurements with added antimicrobial agents.

In another embodiment, the resonant Raman peaks due to lycopenes are monitored. When using a laser of 532 nm wavelength, lycopenes have resonantly enhanced Raman peaks at 1510 to 1520 cm⁻¹ (i.e., 1516 cm⁻¹), 1150 to 1160 cm⁻¹ (i.e., 1156 cm⁻¹), and 1005 cm⁻¹. Further, lycopenes are efficient scavengers of free radicals, and free radicals are always generated by bacteria and fungi cells during normal metabolism and by human cells. Such free radical production is a consequence of chemiosmosis and the generation of ATP during cellular respiration. Thus, the presence of viable actively replicating bacterial and fungal cells in a serum sample will result in free radical production (which is much more amplified compared to radicals from terminally differentiated human cells), which in turn will be scavenged by the serum lycopene. Scavenging these radicals by lycopene will change the optical properties of the lycopene molecule, including its resonant Raman optical properties. These changes in optical properties, such as a decrease in intensity for the lycopene peaks, can be monitored for detecting pathogen viability.

In another embodiment, pathogen viability is monitored (via any of the methods listed above) as selective media are added to the clinical sample. Addition of selective media that favor pathogen growth will result in an increased viability signature, which can be used to classify the pathogen. Addition of selective media that precludes certain pathogen growth will result in a decreased viability signature, which can also be used to identify the pathogen.

In yet another embodiment, free radical production (via a marker, such as the lycopene marker described above) is monitored for human cells under the suspected presence of foreign substances If certain foreign substances are present, such as a pathogen, then the human cells will generate free radicals, which is detected by the Raman instrument.

### Definitions

For the purposes of the present discussion, Raman scattering is any method whereby light incident on a sample at a fixed wavelength is scattered at other wavelengths by an incoherent process due to the absorption of the incident photon by the excitation of the structure from an initially lower (the ground state) to a higher vibrational level, and subsequent relaxation down to a different ground state level.

For the purposes of the present discussion, a Raman band is the spectral profile (intensity versus frequency) corresponding to the Raman scattering from a particular chemical bond within a molecule. It is understood that each chemical bond manifests as a Raman band at distinct frequencies and that in some cases, these Raman bands may overlap, making them difficult to distinguish. Further, it is understood that the Raman cross section of a chemical bond is a constant that defines the intensity of the corresponding Raman peak. Furthermore, it is understood that this cross section can change with wavelength and/or with resonance. Such a resonance change occurs during resonant Raman enhancement.

For the purposes of the present discussion, it is understood that the Raman spectrum of a sample is the sum of all the Raman bands, and the relative heights on individual Raman bands in a Raman spectrum is proportional to the relative abundance of the corresponding chemical bonds multiplied by their Raman cross section.

For the purpose of the present discussion, absorption is any method wherein incident light is absorbed by a sample of interest and the incident photon can interact with structure by any number of mechanisms, including the excitation of outer electrons (corresponding to the absorption of UV or visible radiation), or the excitation of the molecule into higher vibrational/rotational energy states.

Resonant Raman scattering is a process that is understood to be a special type of Raman scattering process that involves the excitation of a molecule from an initial ground state to a real excited state that corresponds to a real vibrational state. Thus, for the purpose of the present discussion, resonant Raman enhancement (or resonance Raman) is any method whereby the Raman cross section of a particular band is enhanced by the strong optical absorption.

For the purpose of the present discussion, a siderophore is a protein released by a pathogen that strips iron from the host's iron-protein complex, and transports the iron to the pathogen.

For the purpose of the present discussion, iron sequestration is the process by which an invading pathogen acquires iron from the host's iron-protein complex. Iron acquisition, in turn, refers to the process by which the host reduces the amount of free iron by creating a protein-iron complex.

For the purpose of the present discussion, it is understood that siderophore mediated iron sequestration is one of several possible mechanisms that can be used by pathogens to sequester iron from the host iron-protein complex. Further, it is understood that regardless of the mechanism, the end result is to strip iron from the iron-protein complex in the host.

For the purpose of the present discussion, it is understood that the different host-protein complexes that can sequester iron in the host include transferrin, lactoferrin, heme and ferritin.

For the purpose of the present discussion, it is understood that the iron-protein complexes can exist in the form that includes iron, or the form that does not include iron. The two forms are described as the ferric and apo-states. Using transferrin as the example, the two forms can be written as Fe-Tr and apo-Tr.

For the purpose of the present discussion, it is understood that carotenoids are compounds that scavenge free radicals, and that lycopene is a very efficient free radical scavenger.

For the purpose of the present discussion, an exomycobactin is an extracellular siderophore (sometimes referred to as a carboxymycobactin) that is used by pathogenic mycobacteria, along with an intracellular mycobactin siderophore, to acquire iron.

For the purpose of the present discussion, minimum inhibitory concentration (MIC) is defined as the minimum concentration of antimicrobial that will inhibit pathogen growth, where the pathogen has a standard concentration (usually defined as 100,000 CFU/mL).

For the purpose of the present discussion, it is understood that the noise in any measurement system is proportional to the square root of the quantity being measured.

### Detection Method Diagrams

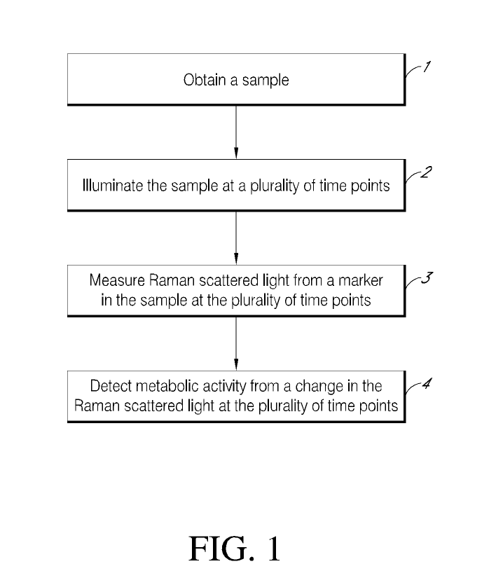

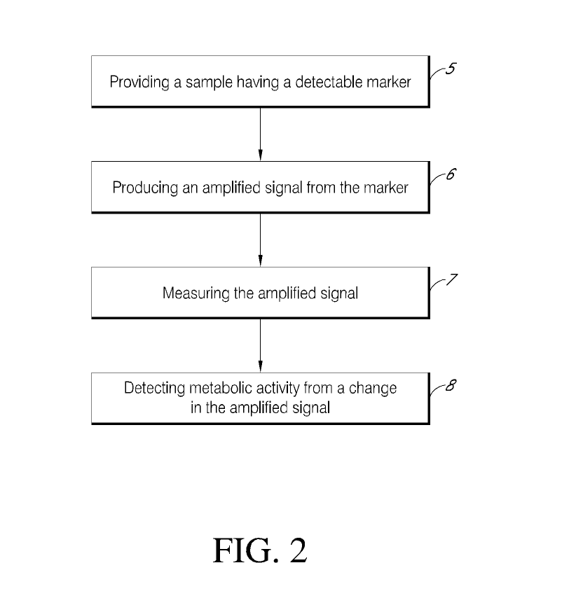

FIGS. 1 and 2 show examples of flow diagrams illustrating methods of detecting metabolic activity in a sample. With reference to FIG. 1, the method begins at block 1 by obtaining a sample. A variety of methods for obtaining a sample are known in the art. In some embodiments, the sample is provided in a form ready for analysis. In other embodiments, the sample requires additional preparation prior to analysis. After a sample is provided at block 1, the method continues to block 2 in which the sample is illuminated at a plurality of time points. The method then continues to block 3 in which Raman scattered light from a marker in the sample is measured the plurality of time points. The method then continues to block 4 in which metabolic activity is detected from a change in the transmitted light at the plurality of time points.

With reference to FIG. 2, the method begins at block 5 by obtaining a sample having a detectable marker. Such a marker is directly or indirectly reflective of metabolic activity. A variety of methods for obtaining a sample are known in the art. In some embodiments, the sample is provided in a form ready for analysis. In other embodiments, the sample requires additional preparation prior to analysis. After a sample is provided at block 5, the method continues to block 6 in which an amplified signal is produced from the marker. One non-limiting example of amplification is resonance Raman enhancement. The method then continues to block 7 in which the amplified signal is measured. Such measurement can occur at one or more time points, including a plurality of time points. The method then continues to block 8 in which metabolic activity is detected from a change in the amplified signal at the one or more time points, including the plurality of time points.

### Detection System Diagram

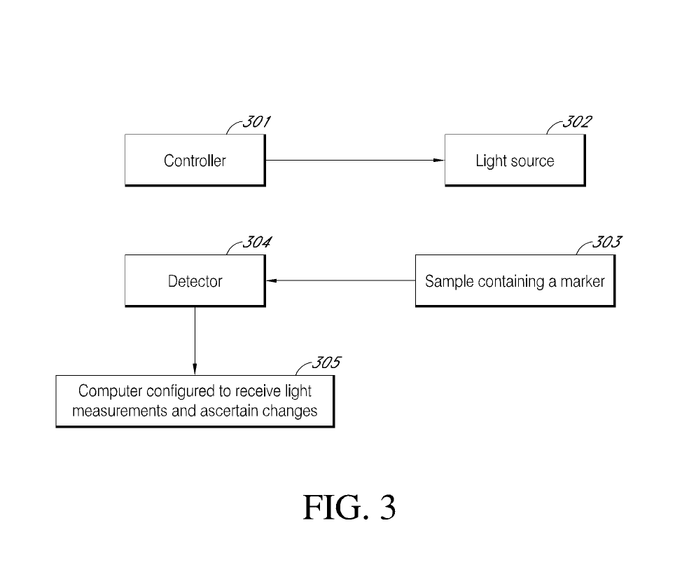

FIG. 3 shows an example of a system for detecting metabolic activity in a sample. With reference to FIG. 3, the system has a controller 301, a light source 302, a sample 303, a detector 304, and a computer 305. Although FIG. 3 represents these system components as distinct blocks, it is understood that in some embodiments one or more system components can function as multiple components. For example, the computer and the controller can be the same component.

With reference to FIG. 3, the controller instructs the light source to illuminate the sample. In some embodiments, the controller allows for periodic illumination. In other embodiments, the controller allows for continuous illumination. The sample 303 contains a marker responsive to metabolic activity in the sample. The marker in sample 303 transmits a signal, such as light, to the detector 304. The detector 304 is configured to measure the signal from the marker. The computer 305 is configured to receive measurements from the detector and ascertain changes in the marker over time. Such changes are indicative of metabolic activity in the sample.

### Description of an Iron Sequestration Method

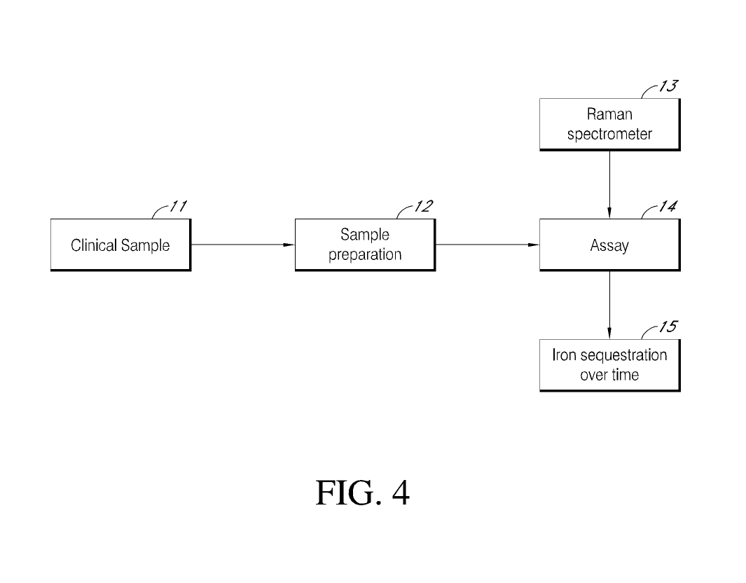

As depicted in FIG. 4, one embodiment comprises the collection of a clinical sample 11, followed by sample preparation 12. The sample preparation 12 converts the sample into the assay 14. The assay 14 is monitored for metabolic activity, which in this embodiment is denoted as iron sequestration over time (15). The metabolic activity is measured using Raman spectrometer 13.

As previously discussed, sample preparation 12 converts the sample into the test assay. The test assay contains all elements necessary of pathogen growth, except for the limited availability of free iron. All iron is sequestered into special iron-protein complexes, and the pathogen must sequester iron from this complex.

In FIG. 4, the sample preparation step 12 comprises the conversion of the clinical sample to one that is conducive to pathogen metabolism except for the non-availability of free iron, with all the iron being sequestered into a host protein (such as transferrin). If the initial clinical sample is blood, then the sample preparation step optionally involves gravity sedimentation and/or centrifuging the blood sample to separate out the serum. The serum will include any pathogens present in the blood, as well as the entire growth medium necessary to support pathogen metabolism except for free iron. If the clinical sample is urine, then the sample preparation step optionally involves the addition of a small urine sample to a suitably prepared stock serum.

Pathogen concentration can be extracted from the rate at which the pathogens sequester iron. At higher pathogen concentrations, the slope of the trace Fe-Tr peak height versus time is greater. Thus, in some embodiments the rate of iron sequestration is pre-characterized with a set of known standard samples of varying pathogen concentrations, and in some embodiments the rate of iron sequestration from the clinical sample is matched against these rates.

As described further herein, antimicrobial susceptibility can be characterized via the metric minimum inhibitory concentration (“MIC”). A series of assays of increasing antimicrobial concentrations is prepared from the same clinical sample. In some embodiments, anti-pathogenic substances of increasing concentration as added to the sample. Monitoring these samples for metabolic activity afford information regarding the effectiveness or ineffectiveness of the anti-pathogenic substance. Additionally, the monitoring of these samples affords information regarding the MIC of an effective anti-pathogenic substance. As one non-limiting example of such an embodiment, the assay in which the iron sequestration marker's trace becomes invariant with time is the minimum inhibitory concentration at the pathogen concentration in the clinical sample.

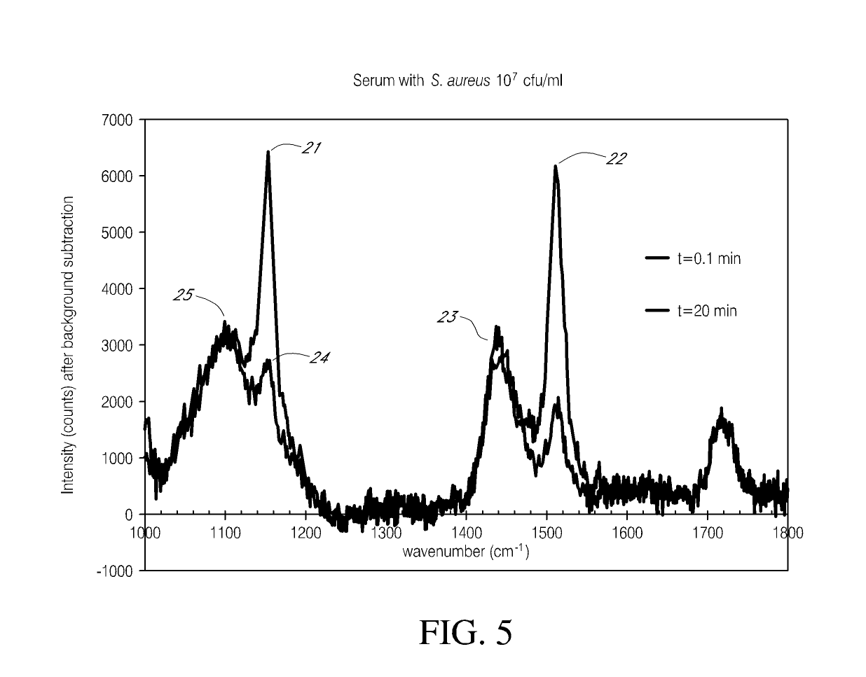

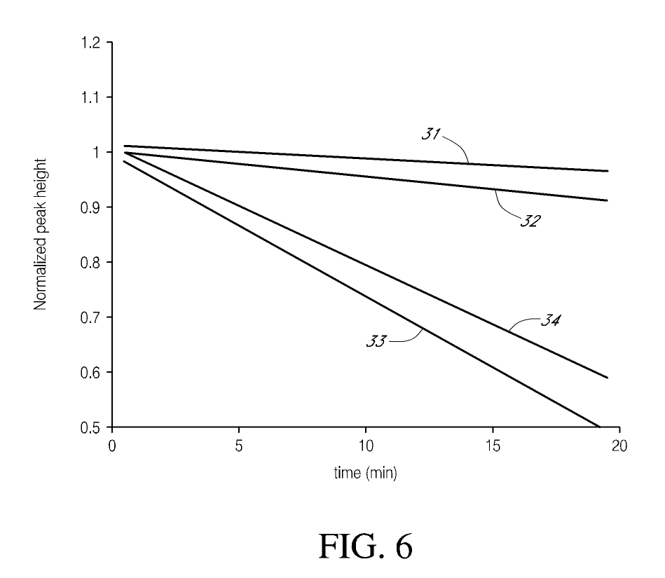

As described above, if the iron sequestration process is monitored via Raman spectroscopy, then the test involves the collection of a series of Raman spectra over time. A hypothetical depiction of this collection for a non-iron marker is depicted in FIG. 5. The individual Raman spectra comprise one or more peaks such as 22, 22, and 23 that, in the case of iron sequestration, are ascribed either to Fe-Tr (21 and 22) or to the apo-Tr and/or serum lipoproteins (23). The peak heights from one of the Fe-Tr peaks (for instance, 22) are monitored over time, as depicted in FIG. 5. If viable pathogens are present, and are sequestering iron to afford Fe-Tr, then the iron-transferrin peaks decrease over time, as depicted by traces 33 and 34 in FIG. 6. On the other hand, if viable pathogens are not present, or if viable pathogens are present in the assay together with an effective antimicrobial agent, then the peak heights are nearly invariant with time. The four traces depicted in FIG. 6 are representative of serum from an uninfected patient (trace 31), serum with a high dose of methicillin resistant Staphylococcus aureus (MRSA, trace 33), serum with MRSA and with an effective dose of vancomycin (trace 32), and serum with MRSA and an ineffective dose of ampicillin (trace 34).

In some embodiments, signal quality is enhanced as a function of time. For example, as pathogens metabolize and consume iron, the level of iron depletes steadily. While the rate of iron depletion may be small for low pathogen concentrations, this depletion can build up to significant levels over time. Thus, within a reasonable time interval of around 20-30 minutes, very low pathogen concentrations are capable of being characterized.

### Pathogen Identification via Siderophores

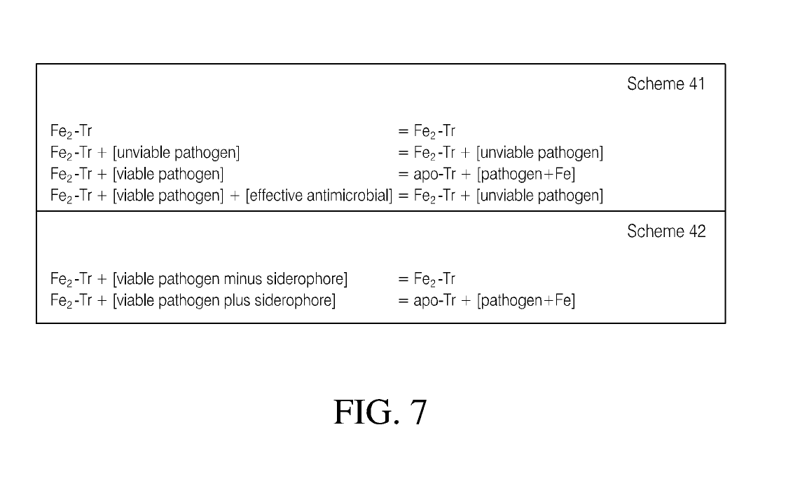

Several addition embodiments can be derived from the basic construct described above. The marker can be summarized by the schematic in FIG. 7. The baseline marker is an iron-protein complex (FIG. 7 describes an iron-transferrin complex, but other proteins can also be used), and is described by Scheme 1 in FIG. 7. If viable pathogens are present, and are sequestering iron from the iron-protein complex, then the corresponding level of the iron-protein complex is reduced. This reduction is monitored via various analytical methods. In some embodiments, the analytical method is spectroscopic, such as Raman spectroscopy. In other embodiments, the analytical method is optical.

In some cases, viable pathogens will not be able to sequester iron from the iron protein complex. One specific example of this is that of pathogenic mycobacteria. In such cases, the marker can be exploited in a device that diagnoses the presence (or absence) of that pathogenic mycobacteria in the clinical sample. The modified marker is described by Scheme 2. Pathogenic mycobacteria require two siderophore proteins that must work in tandem, in order to acquire iron from the host protein complex. These two siderophores include an intracellular mycobactin siderophore, and an extracellular exomycobactin siderophore. Of these, the pathogenic mycobacteria can produce the mycobactin siderophore as needed, but it produces the exomycobactin siderophore only when it is subject to very prolonged conditions of iron deprivation.

Accordingly, when the pathogenic mycobacteria are present in a body fluid, such as serum, it is normally not able to sequester iron from the iron-protein complex, and is thus not able to grow. Serum is said to be bacteriostatic against pathogenic mycobacteria. However, if the exomycobactin is added to the assay sample, then all conditions of iron sequestration are met, and the pathogenic mycobacteria begin iron sequestration. Accordingly, some embodiments involve the addition of the substances to a sample that facilitate pathogen metabolic activity, such as the described exomycobactin siderophore, and the comparison of the resultant metabolic activity (i.e., iron sequestration rate) with the metabolic activity (i.e., iron sequestration rate) in an optional control assay (one without the added exomycobactin). If the iron sequestration rate in the test assay (one with added exomycobactin) is greater than in the control assay (one without added exomycobactin), then that is a positive indicator for the presence of pathogenic mycobacteria. In some embodiments, the comparison with a control assay is optional. In other embodiments, the addition of a substance to the sample that facilitates pathogen metabolic activity can be used to identify and/or speciate the pathogen in the sample.

### Pathogen Detection via Free Radical and Proton Production

In some embodiments, the presence of a pathogen is confirmed by pathogen-associated metabolic activity via changes in the resonant Raman spectra associated with marker redox activity. The presence of pathogens in the test assay results in the generation of free radicals and protons. Free radical/proton production is a guaranteed consequence of cell metabolism, as per the Mitchell hypothesis (see Mitchell P. et al., Biochemical Journal 1961; 81:24; Mitchell P., Nature 1961 July; 191:144-148) which has been demonstrated in microorganisms (Mitchell P., Fed. Proc. 1967 September; 26(5):1370-1379). This “hypothesis” is now the proven and accepted mechanism for energy production in microbial cells. Thus, markers responsive to free-radicals and/or proton production are useful for detecting metabolic activity and the presence of pathogens. Such markers include, but are not limited to, antioxidants, free-radical scavengers, ROS and RNS sensitive dyes, and proton sensitive chemicals such as acid-base indicates. Further, carotenoids including lycopene, which is a component of human serum/plasma, is a very efficient free radical scavenger˜it is said to be the most efficient free radical scavenger present in human plasma (see Wassermann A., Molecular Physics 1959 April; 2(2):226-228; Content and isomeric ratio of lycopene in food and human blood plasma 10.1016/S0308-8146(96)00177-X: Food Chemistry; Ermakov I V et al., J. Biomed. Opt. 2005; 10(6):064028-064028). Upon exposure to free radicals and protons generated by bacterial/fungal metabolism, lycopene is protonated to form a carbocation and ultimately reacts to produce beta-carotene and retinal in the organism.

Lycopene has an optical absorption spectrum that includes a reproducible absorption maximum centered at about 532 nm. Upon protonation (along with other chemical reactions), the optical absorption spectrum of lycopene redshifts, and the absorption peak at about 532 nm disappears. The corresponding Raman spectrum of lycopene is resonantly enhanced when collecting Raman spectra with an incident laser of about 532 nm wavelength. By contrast, when lycopene is protonated, and the absorption maxima at about 532 nm redshifts, the resonant Raman enhancement also decreases. These properties result in the following observations about the optical properties of human serum or plasma: (1) when collecting Raman spectra with a laser of 532 nm wavelength, the Raman spectra of human serum (or plasma) is dominated by that of lycopene (dominant peaks at 1516 cm⁻¹ and 1156 cm⁻¹), even though lycopene is nominally not the dominant component of human serum. (2) If viable pathogens are present, and if those pathogens are undergoing metabolic activity, then the intensity of the lycopene peaks in the Raman spectra decrease over time.

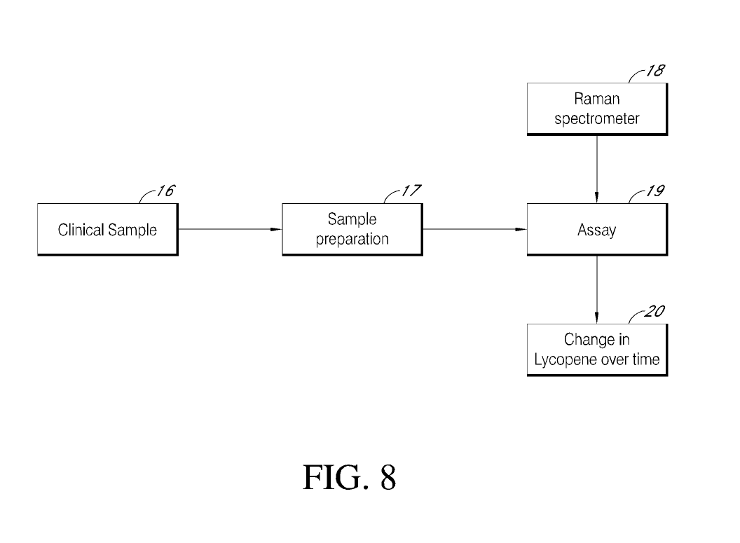

Such an embodiment is described in FIG. 8. With reference to FIG. 8, a clinical sample 16 is obtained and optionally subjected to sample preparation 17 to afford a sample that is subjected to assay 19. The assay 19 includes illuminating the sample in the assay with Raman spectrometer 18 and detecting a change in the amount of lycopene over time, as illustrated in block 20. Such changes, as measured by signals from the assay, are indicative of metabolic activity. The metabolic activity, in turn, is indicative of the presence of pathogen(s). In one embodiment, the presence of pathogen is detected via the free radicals they produce during metabolism. While the metabolism of all living organisms results in a concentration of free radicals at the cell wall, bacterial and fungal pathogens have a single cell wall. Thus, metabolism in fungal and bacterial pathogens produces free radicals concentrated at their cell walls, where these free radicals can be scavenged by free radical scavengers present in serum. Some of the free radical scavengers have a resonantly enhanced Raman spectrum. For instance, and as described above, lycopene is a red-carotenoid similar to beta-carotene; and which has a strong absorption spectrum at 532 nm. Thus, when using a 532 nm laser, lycopene has a resonantly enhanced Raman spectrum that dominates the other intrinsic components of serum. Further, lycopene is a very efficient free radical scavenger, and reacts with the free radicals produced by pathogen metabolism. The resultant change in lycopene structure can be easily monitored by Raman spectroscopy using a laser of about 532 nm wavelength. Those skilled in the arts will recognize that this basic formulation can be extended into other formulations that are similar. For instance, the basic principles can be applied to the detection of beta carotenes (which are produced by some pathogens during their metabolism) when using a Raman scattering setup with a slightly lower laser wavelength of 480-510 nm. Those skilled in the arts will also recognize that a combination of lasers can be used to develop a biochemical profile of the pathogen. For instance, one can use a Raman instrument with laser wavelength of 488 nm to characterize beta-carotene production, and combine that with a Raman instrument of laser wavelength 532 nm to characterize lycopene-free radical scavenging. The resultant biochemical profile characterizes beta-carotene production during the metabolism cycle, and can be used to identify the pathogen.

In some embodiments, the rate of marker consumption is proportional to the amount of pathogens in the sample. For example, in some embodiments, the rate of lycopene peak intensity decreases over time in proportion to the amount of pathogens present in the sample, and with the rate at which those pathogens are metabolizing. Consequently, in some embodiments, the rate of marker consumption is used to determine the concentration of pathogens in a sample.

With reference to FIGS. 5 and 6, if the lycopene consumption is monitored via Raman spectroscopy as an indicator of metabolic activity, then the test involves the collection of a series of Raman spectra over time. A representative depiction of this collection is depicted in FIG. 5. The individual Raman spectra comprise one or more peaks such as 22, 22, and 23 that, in the case of lycopene consumption, are ascribed either to lycopene or other constituents in the sample. If viable pathogens are present, lycopene concentration will decrease and the lycopene peak will decrease over time, as depicted by traces 33 and 34 in FIG. 6. On the other hand, if viable pathogens are not present, or if viable pathogens are present in the assay together with an effective antimicrobial agent, then the peak heights are nearly invariant with time as depicted in FIG. 6. As previously described, the four traces depicted in FIG. 6 are representative of serum from an uninfected patient (trace 31), serum with a high dose of methicillin resistant S. aureus (MRSA, trace 33), serum with MRSA and with an effective dose of vancomycin (trace 32), and serum with MRSA and an ineffective dose of ampicillin (trace 34).

Additionally, certain pathogens produce carotenoids as a sign of metabolic activity. In such cases, traces using a carotenoid as a marker (i.e. lycopene) that demonstrate the presence of pathogens will have a positive slope over time because the concentration of the marker in the sample is increasing with the pathogen's metabolic activity. However, traces demonstrating the absence of pathogens will still remain relatively constant as the peak intensity remains nearly invariant. Thus, it is the change in peak intensity over time for a marker, representing metabolic activity in the sample, which is indicative of the presence of metabolic activity and a pathogen. Such change can be either marker consumption or marker production, discussed in more detail below.

### Detecting Metabolic Activity via Marker Production

In some embodiments, metabolic activity in a sample is detected by the production of a marker. Examples of the production of a marker in a sample include an element-pathogenic protein complex (i.e., Fe-Tr), a reduced antioxidant, a reduced free radical scavenger, a reduced carotenoid, or even a reduced lycopene. Thus, metabolic activity in a sample is detectable from a decrease in the amount of a marker (such as the marker's consumption), or metabolic activity is detectable from an increase in the amount of a marker. As a non-limiting example of these concepts, lycopene could be a marker and its consumption in a sample would indicate metabolic activity. Conversely, the product of lycopene functioning as an anti-oxidant could be a marker and its production would indicate metabolic activity.

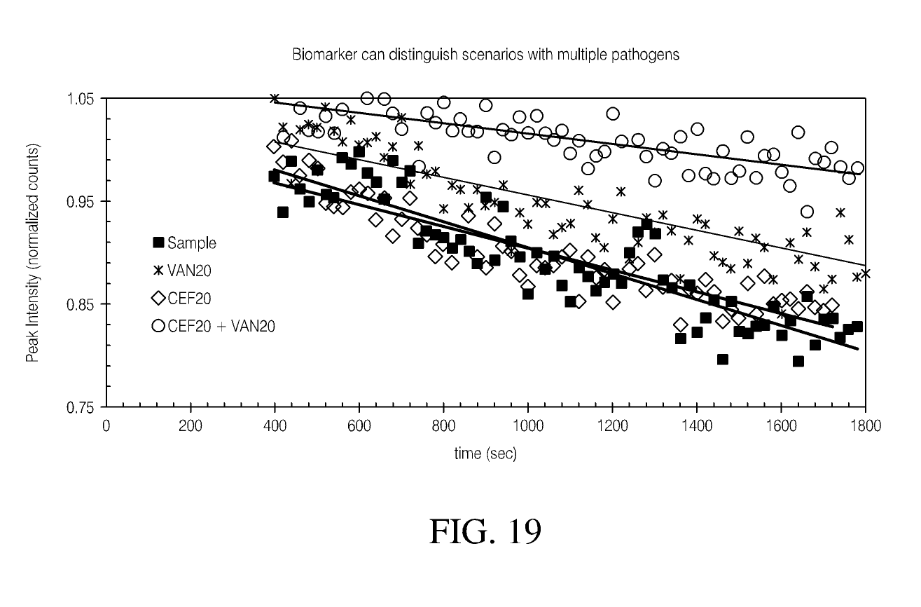

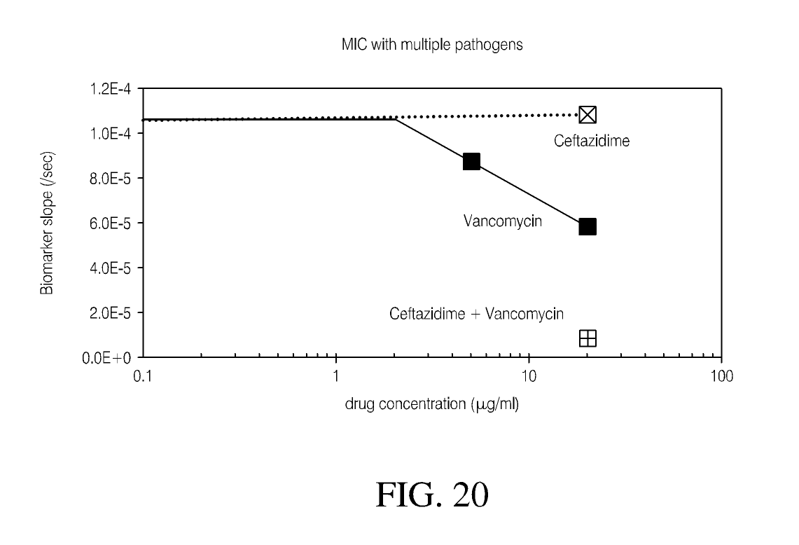

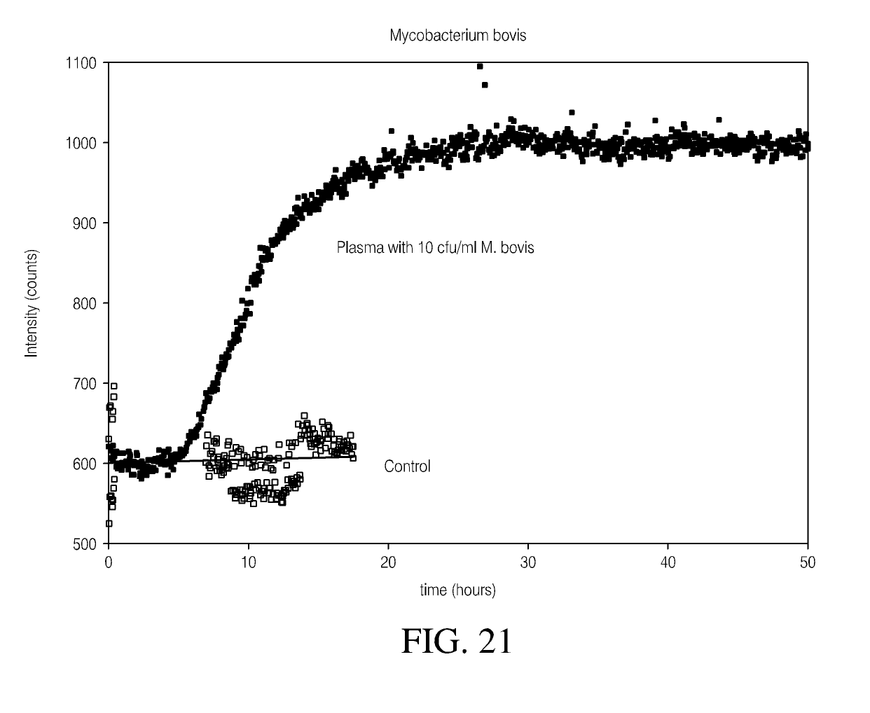

In some embodiments, pathogens produce the marker. For example, some pathogens produce carotenoids, and specifically lycopene, and this production can be detected to indicate metabolic activity in a sample, the presence of a pathogen in a sample, and even facilitate classification of the pathogen in a sample. Indeed, carotenoid and lycopene production have been documented in several mycobacteria species including, but not limited to M. phlei, M. kansasii, and M. aurum. Additionally, certain embodiments described herein have been used to detect lycopene production in M. bovis (See FIGS. 19 and 20) and M. fortuitum (See FIG. 21). Moreover, carotenogenesis in M. marinum and other mycobacteria sp. is known and found to be light-dependent. Finally, M. tb is known to mediate a carotenoid oxygenase; and different mycobacteria species can be phenotyped based on their carotenoid production. Thus, in some embodiments pathogens, and specifically mycobacteria, are detected from the production of carotenoids in the sample. In other embodiments, pathogens, and specifically mycobacteria, are detected from the production of lycopene in the sample.

Antimicrobial Susceptibility and/or Pathogen Identification via Antimicrobial Susceptibility

In some embodiments, the susceptibility of pathogens to anti-pathogenic substances is determined. For example, the susceptibility of a particular bacterium to a particular anti-bacterial and/or anti-bacterial cocktail is determined. Similar susceptibilities are also easily determined for particular fungi and viruses in relation to anti-fungals and anti-virals. Additionally, in some embodiments, pathogens in the sample are unknown and susceptibility is determined independently of knowledge regarding the identity of the pathogens. Thus, the likely effectiveness or ineffectiveness of a particular treatment is readily determined in some embodiments. Moreover, some embodiments provide for the rapid determination of the minimum inhibitory concentration (MIC) metric.

MIC is the lowest concentration of drug that effectively inhibits in vitro growth of the target organism. With standard MIC testing, the test requires pure growth of the organism under study and usually becomes available to clinicians at least 48-72 hours after a sample is collected. In some embodiments, however, and because markers such as lycopene reflect metabolic activity and metabolic rate is expected to decrease when an effective anti-pathogenic drug acts on the pathogen, the MIC metric is able to be characterized faster than standard testing time. In some embodiments, the MIC metric is generated within about 30 minutes of testing.

Some embodiments determine the effectiveness/ineffectiveness of a particular anti-pathogenic substance in samples infected with unknown (pathogens) and/or one or more known pathogens. In other embodiments, the effectiveness/ineffectiveness of a combination of more than one anti-pathogenic substance is determined for a sample with unknown pathogen(s) and/or one or more known pathogens.

### Detection of Toxins in a Sample

One effect of a toxin on a mammalian cell line is the induction of metabolic stress. Free radicals are produced as a consequence of metabolic stress, and therefore mammalian cells should produce free radicals upon exposure to toxins. Carotenoids, especially lycopene, are efficient scavengers of free radicals. Thus, the exposure of mammalian cells to toxins should result in measurable changes in lycopene concentration. Such changes are detectable using various methods described herein.

In some embodiments, a marker is used to detect the presence of chemical or biological toxins in samples such as blood, urine or other clinical samples. Other clinical samples include, but are not limited to, samples prepared specifically for testing a substance to determine if it is toxic and/or toxic concentrations. In some embodiments, the marker is an antioxidant, a free-radical scavenger, a carotenoid, and/or lycopene.

Using a blood sample as an example, the chemical or biological toxin is a small molecule or cell and it will be concentrated in the serum under normal processing methods such as centrifugation or gravity sedimentation. Mammalian cells are combined with the serum or portion thereof to afford a sample. A nutritional broth is optionally included in the sample. If a chemical or biological toxin is present from the sample, then it will induce free radical production by the mammalian cells. Being responsive to free radicals, the marker will undergo a measurable change and provide for the detection of a toxin in the sample.

In other embodiments, the marker is lycopene and human cells, such as HL60 cells, are utilized. If substances toxic to human cells are present in the sample, the human cells will generate free radicals. The scavenging of the free radicals by the lycopene marker will result in a measurable change in the lycopene marker or a signal representative of a change. In some embodiments, the measured changes are detected with a Raman instrument.

In some embodiments, one or more substances are tested to determine if they are toxic to human cells. Indeed, such substances can be combined with human cells, along with optional nutritional broth, and the effect of the substance on the human cells can be monitored. Thus, toxicity of a substance (or the presence of a toxin in the sample) is detected by free radical production and a change in a marker sensitive to free radicals. In some embodiments, samples are tested for presence of one or more bacterial toxins, fungal toxins, and/or chemicals (i.e. organic, inorganic, and organometallic compounds, including solvents and reagents used in the synthesis of such compounds). In other embodiments, one or more substances such as chemicals (i.e. organic and inorganic compounds, including solvents and reagents used in the synthesis of compounds) are included in a sample to determine if a substance is toxic. In some embodiments, various concentrations of a substance are tested to determine likely threshold toxicity values. For example, pesticides, pharmaceuticals, pharmaceutical ingredients, active pharmaceutical ingredients, carriers, fillers, synthetic intermediates, and impurities identified in pharmaceuticals represent substances whose toxicity or lack thereof is of particular interest.

In some embodiments, the measured changes and/or signals accumulate over time and enable the detection of very low toxin levels. In some embodiments, the detection limit is 1 μM, 5 μM, 10 μM, 20 μM, 30 μM, 40 μM, 50 μM, 60 μM, 70 μM, 80 μM, 90 μM, and/or 100 μM, and/or a range bounded by any two of the aforementioned numbers, and/or about any of the aforementioned numbers. In other embodiments, the limit of detection is about 4 μM. In other embodiments, the detection limit is less than 1 μM, 5 μM, 10 μM, 20 μM, 30 μM, 40 μM, 50 μM, 60 μM, 70 μM, 80 μM, 90 μM, and/or 100 μM, and/or less than a range bounded by any two of the aforementioned numbers, and/or less than about any of the aforementioned numbers.

Measurement with Enhanced Sensitivity and Resolution

From the viewpoint of a measurement system, noise is proportional to the square root of the quantity being measured. Previous antimicrobial susceptibility tests have measured a variable that is directly proportional to the amount of pathogen present in a clinical sample, and this approach results in a large measurement error. For instance, U.S. Pat. No. 4,448,534 describes an apparatus for antibiotic susceptibility testing wherein the bacteria count in a multi aliquot tray is determined by optical density methods. Because the baseline measurement includes a large number of pathogenic cells, and the error in the measurement is proportional to the square root of the number of pathogenic cells, the effects of the antimicrobial drug can be discerned only when the drug has reduced the viability of a large number of cells. This approach results in a large time requirement for the testing.

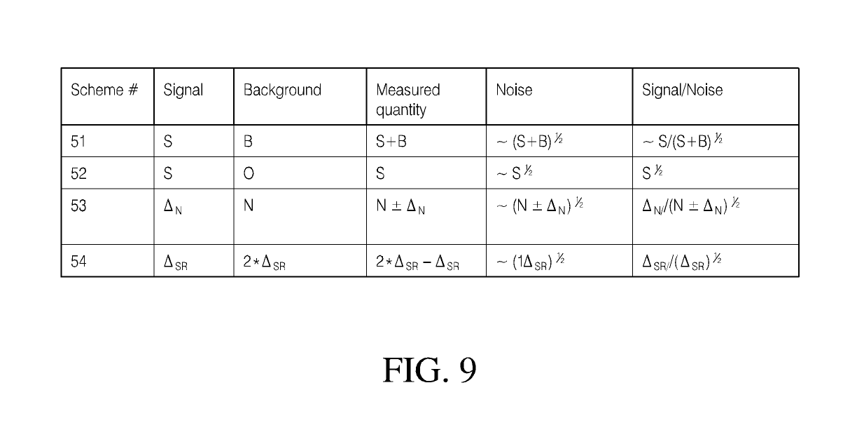

As illustrated in FIG. 9, to a first approximation the noise in any measurement system is proportional to the square root of the noise being measured. Scheme 51 and 52 illustrate the signal to noise ratios for a measurement system that measures a signal S with and without a background level B. Previous methods to measure antibiotic susceptibility can be summarized by Scheme 53, where N represents a variable that is proportional to the bacterial cell count, and Δ_{N} is the change in that variable. Under these circumstances, the measured quantity is N±Δ_{N} and the noise in the measurement becomes the square root of N±Δ_{N}. Thus, the measured signal rises above the noise threshold only when Δ_{N} becomes comparable to N. This requirement generally translates into isolating the bacterial cells from the clinical sample, such that the doubling time of 20 minutes will create a sufficiently strong signal. Alternatively, if the device is working directly with the clinical sample, then the method must wait for several doubling times, such that the bacterial cell count exceeds the count of the intrinsic clinical sample components.

By contrast, embodiments disclosed herein measure a host-pathogen interaction that is either present (when viable pathogens are present), or absent (when viable pathogens are not present, or when their activity has been suppressed by an effective anti-pathogenic substance, such as an antimicrobial). Since the baseline measurement in some embodiments is the absence of an interaction, the measurement sensitivities are much greater, even when the time required for testing is small. More generally speaking, certain embodiments measure a scarce resource associated with pathogen presence (i.e., iron or lycopene). The benefits of this can be described by Scheme 54 in FIG. 9. The signal is Δ_{SR}, which is the change in the level of the scarce resource. This signal is measured on a relatively small background (2*Δ_{SR} is the example cited in FIG. 9, it can be any other number that is not much greater than Δ_{SR}). Thus, in some embodiments, the noise is proportional to the square root of Δ_{SR}.

To varying degrees, all previous approaches for antimicrobial susceptibility testing suffer from this inherently flawed approach that was described for U.S. Pat. No. 4,448,534. For instance, U.S. Pat. No. 6,379,920 describes a method whereby the Raman spectra of a clinical sample from a non-infected patient are used as a reference that is subtracted from the Raman spectra of an unknown clinical sample. With this method, the inventors claim that specific bacteria can be identified sooner and without culturing. However, the baseline measurement is the Raman spectra of the unknown clinical sample, and contains all the intrinsic components of the clinical sample. Further, the Raman bands of those intrinsic components overlap with the Raman bands of the bacterial pathogens; thus the bacterial cell count must be very large before a significant differential measurement can be made.

U.S. Pat. No. 3,983,006 describe a method for determining minimum inhibitory concentration (MIC) of an antibiotic by continuously measuring the change in optical properties in response to the bacterial growth rate of a bacterial suspension in the absence and the presence of the antibiotic. As with U.S. Pat. No. 4,448,534, this method suffers from a large measurement error associated with the measurement of a large number of pathogenic cells, consequently the device requires a large timescale for the antimicrobial to kill a large number of bacterial cells before an effective measurement can be made.

### Enhancement Due to Raman Amplification

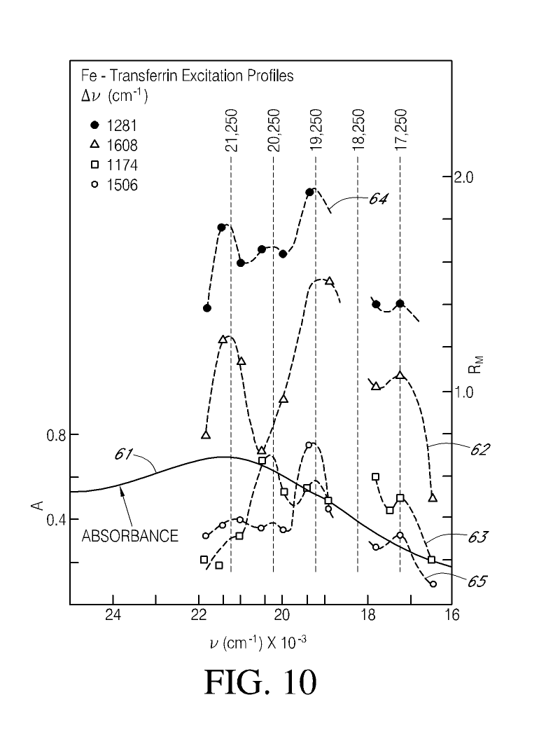

Some embodiments exploit various resonant processes to amplify the measured signal. This feature is illustrated with the scheme outlined in FIG. 10. Specifically, important optical differences exist between Fe-Tr and Tr. For instance, Fe-Tr has a broad optical absorption peak centered at a wavelength of 485 nm. Because of this optical absorption peak, various Fe-Tr Raman bands display a strong resonance Raman enhancement when the Raman laser wavelength is located within this optical absorption band. FIG. 10 illustrates the relationship between the optical absorption 61, the Raman cross sections of 4 Fe-Tr peaks at 1608, 1506, 1281 and 1174 cm⁻¹ (62, 63, 64 and 65, respectively) and the wavelength. FIG. 10 demonstrates that if the laser wavelength is located at a value at which one or more of the Fe-Tr peak is resonantly amplified, then the resultant Raman spectrum will be dominated by the spectrum of Fe-Tr. Moreover, resonance Raman enhancement is not limited to the Fe-Tr system described above, but the amplification is present for other markers as well. For example, resonant Raman enhancement is available for markers such as anti-oxidants, free radical scavengers, and/or carotenoids (i.e., beta-carotene and lycopene). Indeed, the Raman signature of lycopene is resonantly enhanced by the use of 532 nm light. This resonant enhancement can be of the order of about 10 times to about 1000 times. In some embodiments, resonant enhancement is of the order of 10 times, 50 times, 100 times, 200 times, 300 times, 400 times, 500 times, 600 times, 700 times, 800 times, 900 times, and/or 1000 times, and/or a range bounded by any two of the preceding number, and/or about any of the preceding numbers. Thus, resonance Raman enhancement allows for the testing of the clinical sample without requiring any isolation or growth of the bacteria. In some embodiments, resonance Raman is used to amplify the measured signal. In other embodiments, amplification is used without needing to isolate the pathogen in the sample. In some embodiments, amplification is used without needing to allow the pathogen count time to increase. In other embodiments, amplification is used without needing to concentrate the pathogen in the sample.

In some embodiments, the marker “integrates” over time such that changes in the measured signal are cumulative. Some markers, however, are “differential.” One example of an “integrative” marker is lycopene, whereas a fluorescence biomarker is one example of a “differential” marker. To understand the distinction, consider a small number of bacterial cells being simultaneously studied by lycopene Raman and a fluorescence biomarker. Assuming that the number of cells does not change significantly during the measurement, the output of the fluorescence marker is proportional to the rate of metabolism of that group of cells, which is likely a constant if the conditions do not change. In contrast, however, the output of the lycopene marker is proportional to the total metabolic activity, which is the time integral of the rate of metabolism; and which therefore changes steadily with time. Thus, after a sufficiently long time has elapsed, the lycopene marker will have a value that is significantly different from the initial value, and which can be read relatively easily above the measured noise.

### Pathogen Classification Using Selective Media

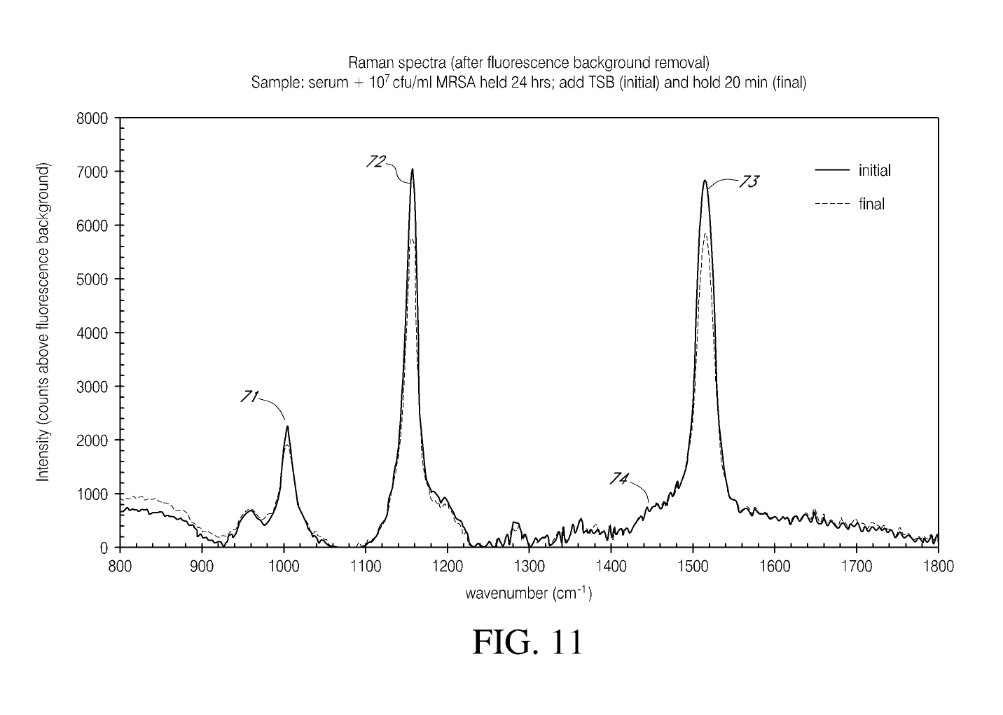

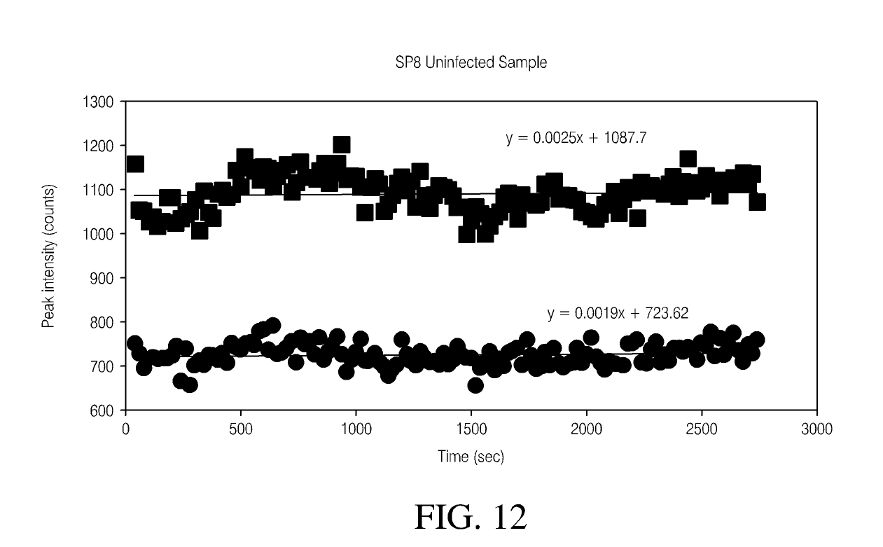

In some embodiments, an anti-pathogenic substance is present in the sample. One example of an anti-pathogenic substance is a selective media, or culture broth. In some embodiments, selective media is used to identify the pathogen in the sample. FIGS. 11, 12 and 13 further illustrate identification using selective media. FIG. 11 illustrates the Raman spectrum at different time points for a serum sample that has been inoculated with 10⁷ cfu/mL S. aureus. In the absence of any added broth, the peak heights do not change as a function of time. Thus, in that regard, the behavior of the infected sample without added broth is nearly identical to that of an uninfected sample. FIG. 12 depicts the time profile of a serum sample from an uninfected febrile patient diluted in 80% broth. The two traces depict the peak heights at 1516 and 1156 cm⁻¹, which are both ascribed to lycopene. In this case, the two peak heights remain nearly invariant over time. In the absence of added broth, infected samples (including those from infected patients) demonstrate this behavior˜the peak heights do not change over time.

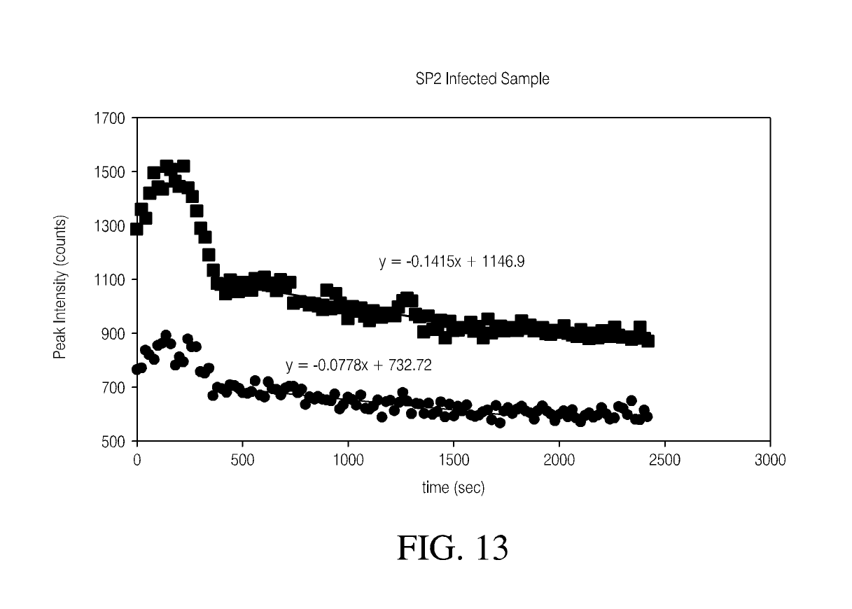

Upon the addition of a medium that favors growth, the peak heights start to decrease. One example of this is depicted in FIG. 13, which depicts the peak heights at 1516 and 1156 cm−1 (which are both ascribed to lycopene) as a function of time for a serum sample from an infected patient, but with the addition of trypticase soy broth (TSB). TSB is a selective media that enables the growth of MRSA. Accordingly, FIG. 13 illustrates a decrease in the Raman peaks at 1516 and 1156 cm⁻¹, as depicted in FIG. 13. This decrease indicates the presence of metabolic activity, and thus the presence of a pathogen.

In some embodiments, the culture broth suppresses the growth of certain pathogens. In other embodiments, the culture broth promotes the growth of certain pathogens. In some embodiments, combinations of anti-pathogenic and pro-pathogenic media are utilized. Thus, addition of substances to the sample can facilitate pathogen identification and/or classification.

Although there is no one nutrient medium that facilitates the metabolism of one specific species of microorganism while suppressing all others, use of a combination of selective media can help us classify the organism. For example, MacConkey medium contains bile salts and the dye crystal violet which inhibits gram-positive organisms and as a result will not support the growth metabolic activity of S. aureus and would promote the metabolism of gram negative bacteria such as K. pneumoniae, A. baumannii and P. aeruginosa (MacConkey Agar Plates Protocols [Internet]. [date unknown] Available from: http ://www.microbelibrary.org/index.php/component/resource/laboratory-test/2855-macconkey-agar-plates-protocols. In contrast, Columbia CNA medium (which includes the antimicrobials colistin and nalidixic acid) selects for gram positive organisms because the included antimicrobials inhibit gram negative bacteria Biol 230 Lab Manual, Lab 3 [Internet]. [date unknown]; Available from: http://faculty.ccbcmd.ed/courses/bio141/labmanual/lab3/lab3.html). Furthermore, the use of mannitol salt (with 7.5% salt) would facilitate the metabolism of S. aureus, inhibit the growth of gram negatives and importantly inhibit growth of S. epidermidis (a commensal most commonly responsible for contaminating wound cultures) Microbiology.Media.Tests.Pictures.pdf [Internet]. [date unknown]; Available from: http://www.delta.edu/files/Microbiology/Microbiology.Media.Tests.Pictures.pdf).

Another example is the vancomycin-resistant Enterococcus. These organisms can be selected by the use of bile esculin medium which supports only the growth and metabolism of enterococci and none of the rest of the above mentioned multidrug resistant (“MDR”) bacteria since bile inhibits the growth of other gram positive organisms including MRSA and contains sodium azide which inhibits the growth of gram negatives (Microbiology Lab□: MOLB 2210 [Internet]. [date unknown]; Available from: http://www.uwyo.ed/molb2210_lab/info/biochemical_tests.htm#bile). Differentiation between gram negative MDR organisms can be done by the use of PC medium (Campbell M E, Farmer S W, Speert D P. New selective medium for Pseudomonas aeruginosa with phenanthroline and 9-chloro-9[4-(diethylamino)phenyl]-9,10-dihydro-10-phenylacridine hydrochloride (C-390). J. Clin. Microbiol. 1988 September; 26(9):1910-1912) which specifically supports the metabolism and growth of P. aeruginosa and not the other two gram negative rods. By contrast, Leeds Acinetobacter medium (“LAM”) will support the growth and metabolism of A. baumannii but not the growth of P. aeruginosa or K. pneumoniae (Jawad A, et al. Description of Leeds Acinetobacter Medium, a new selective and differential medium for isolation of clinically important Acinetobacter spp., and comparison with Herellea agar and Holton's agar. J. Clin. Microbiol. 1994 October; 32(10):2353-2358).

Thus, and to summarize, in some embodiments, a multi-step approach with selective media is used to subclassify the pathogen. The steps include one or more (or a combination) of the different select media described in the paragraphs above.

Implementation with IR Absorption Spectroscopy Methods

In some embodiments, the method for detecting metabolic activity is performed with infra-red (“IR”) absorption spectroscopy methods. IR absorption arises from vibrational bands that are identical to the Raman bands. Thus, the IR signature of a molecule tends to be similar to its Raman signature. However, IR absorption methods cannot exploit absorption enhancement like Raman methods in order to maximize the detection of metabolic activity (i.e., the differences between apo-Tr and Fe-Tr as described previously). Consequently, IR absorption methods produce similar baseline spectra during analysis. For example, the baseline infra-red spectrum of Fe-Tr tends to be similar to the baseline infra-red spectrum of apo-Tr. However, metabolic activity (such as the apo-Tr and Fe-Tr signatures) can be detected and/or differentiated by simultaneously irradiating the two samples with a laser light centered at the wavelength of resonant absorption enhancement˜doing so alters the corresponding infrared absorption peaks in much the same way that it alters the Raman bands.

### Additional Embodiments

Other embodiments provide for a method for characterizing the state of respiration of a human, or pathogenic cell; wherein the method comprises monitoring the production of free radicals produced during respiration. In some embodiments, the free radical production is monitored via its effect on free-radical scavengers. The effect on free radical scavengers is monitored via resonant Raman spectroscopy methods. In some embodiments the measured signal is of a quantity that accumulates over time, thereby giving a larger measurement value with lower noise.

Other embodiments provide for a method for characterizing the state of respiration of a human, or pathogenic cell; wherein the method comprises monitoring the production of carotenoids produced during respiration. In some embodiments, the production of carotenoids is monitored via resonance Raman spectroscopy. In some embodiments the measured signal is of a quantity that accumulates over time, thereby giving a larger measurement value with lower noise.

In some embodiments, the method is implemented with a laser of wavelength 1, which gives one marker for pathogen metabolism; and with another laser of wavelength 2, which gives another marker for pathogen metabolism; and the combination of the two markers to develop a biochemical profile of the pathogen. In other embodiments, the method is applied to the identification of the pathogen. In some embodiments, the method is applied to the detection, characterization and quantification of pathogens present in a clinical sample. In other embodiments, the method is applied to the detection, characterization and quantification of chemical or biological toxins present in a clinical sample.

Other embodiments provide for a method for characterizing pathogenic cells present in a clinical sample, wherein the method comprises monitoring the rate at which the pathogens consume, generate, and/or modify a scarce resource in the clinical sample. In some embodiments, the measurement of the scarce resource is performed by methods that intrinsically amplify the signal due to that scarce resource. In other embodiments, the consumption or production of a scarce resource is mapped as a function of various selective media that are added to the assay, and the results are used to identify the pathogen. In some embodiments, the consumption or production of a scarce resource is mapped as a function of added antimicrobial agents, and the results are used to develop antimicrobial drug susceptibility information for the pathogen present in the clinical sample.

In other embodiments, the scarce resource is lycopene and the pathogens consume lycopene by generating free radicals that are scavenged by lycopene. In some embodiments, lycopene consumption is monitored by resonance Raman spectroscopy or by non-resonant Raman spectroscopy. In some embodiments, the scarce resource is beta-carotene and the pathogens produce beta-carotene. In other embodiments, the beta-carotene production is monitored by resonance Raman spectroscopy or by non-resonant Raman spectroscopy.

In other embodiments, the scarce resource is iron that has been sequestered by the host vertebrate into special iron containing proteins. In some embodiments, the iron containing protein is transferrin. In other embodiments, the iron containing protein is lactoferrin. In some embodiments, the iron containing protein is ferritin. In some embodiments, the iron sequestration process is monitored by Raman spectroscopy. In other embodiments, the iron sequestration process is monitored by infrared absorption spectroscopy. In some embodiments, the iron sequestration process is monitored by color titration methods, including UV/visible absorption spectroscopy. In other embodiments, the iron sequestration from the host protein is enabled by the addition of siderophore that is normally not present in the assay. In some embodiments, the addition of siderophores specific to a particular pathogen enables recognition and identification of that pathogen.

In some embodiment the Raman spectroscopy method is amplified by a resonance process, thereby providing for enhanced detection limits and a faster detection time.

Some embodiments provide for a method to characterize the susceptibility (or resistance) of an unknown (or known) pathogen in the clinical sample to an antimicrobial agent. In some embodiments, the pathogen is bacterial. In other embodiments, the pathogen is fungal. In some embodiments, the pathogen is viral.

In some embodiments, the clinical sample is blood. In other embodiments, the clinical sample is urine. In some embodiments, the clinical sample is cerebrospinal fluid. In other embodiments, the clinical sample is sputum.

### Samples:

In some embodiments, samples are obtained in a clinical setting from a patient. In some embodiments, samples include a body fluid. Body fluids include, but are not limited to the following fluids: amniotic fluid, aqueous humour, vitreous humour, bile, blood, blood serum, breast milk, cerebrospinal fluid, chyle, lymph, ejaculate, gastric acid, gastric juice, mucus (including nasal drainage and phlegm), peritoneal fluid, pus, pleural fluid, saliva, sebum, semen, sweat, tears, intra-ocular fluid, secretions, vomit, feces, and urine.

In some embodiments, samples are taken from animals. These samples include body fluids and/or swabs from the animals. In some embodiments, samples are taken from living and non-living objects by swabbing a surface of the object. In other embodiments, portions of an object are taken as a sample.

Thus, in some embodiments obtaining a sample includes collecting the sample from a patient, animal, or object. However, in other embodiments, obtaining a sample includes receiving an already collected and optionally processed sample.

In some embodiments, samples are manipulated after collection and prior to analysis. Such manipulation includes, but is not limited to, the addition of the following: nutrients, anti-pathogenic substances (i.e. culture media, both selective and non-selective; antibiotics; antifungals; and antivirals), pro-pathogenic substances, markers, mammalian cells, substances for toxicity measurements, and/or supplements necessary for a marker to function. Additional manipulation includes routine sample handling procedures, removal of sample constituents (i.e., filtering, centrifugation, and precipitation with or without filtration or centrifugation), sample fixation, and changes in the sample atmosphere (i.e., manipulating gas levels such as oxygen and carbon dioxide; and placing under an inert atmosphere, an aerobic atmosphere, and/or an anaerobic atmosphere). Manipulation may occur before, during, or after sample analysis begins. In some embodiments, the sample is cultured. For example, a sample optionally includes culture broth and/or culture media. In other embodiments, the sample includes cultured cells.

In some embodiments, the sample includes blood and/or blood components. Routine sample handling procedures are known for the collection and manipulation of blood into its components. In some embodiments, blood components such as cells are removed. In other embodiments, blood components such as red blood cells are removed. In some embodiments, both red blood cells and white blood cells are removed. In other embodiments, plasma is obtained for use in the sample. Methods to remove blood components are known in the art. Examples include, but are not limited to, gravity sedimentation and/or erythrocyte sedimentation rate procedures. Gravity sedimentation is optionally performed in the presence of an anticoagulant. In some embodiments, blood components are taken for inclusion in the sample after a complete blood count is performed. In other embodiments, blood components are taken for inclusion in the sample from a complete blood count test. In some embodiments, blood components are taken for inclusion in the sample after an erythrocyte sedimentation rate procedure. In other embodiments, blood components are taken for inclusion in the sample during an erythrocyte sedimentation rate procedure.

As a representative procedure for sample preparation, blood from a patient is drawn into a vacutainer that is coated with an anticoagulant Suitable anticoagulants include ethylenediaminetetraacetic acid (“EDTA”) or citric acid, but EDTA is preferred. The vacutainer is allowed to rest for about 40 minutes (although longer times are optional), during which time the red blood cells settle to the bottle due to gravitational forces. The clear (or pale yellowish) liquid that remains on top is the plasma. The bacterial and fungal cells stay in the plasma layer. Experiments indicate that bacterial/fungal cells in blood substantially retain their viability when the red blood cells are separated by this gravity based method. Advantages of this processing step are that it is consistent with current testing protocol, and does not impose any new sample processing steps. Clinicians are very familiar with the complete blood count (“CBC”) test which involves drawing blood directly into an anticoagulant coated vacutainer. Additional embodiments include a sample processing step that uses a small centrifuge spinning at low enough speeds such that the microorganism loss from the plasma layer is minimized, but which also speeds up the sample processing time (i.e., from about 40 minutes down to about 10 minutes or less).

### Illumination/Light Sources

Light sources and sources for illuminating samples are not particularly limited. In some embodiments, the light source produces light with a wavelength in the UV region. In other embodiments, the light source produces light with a wavelength in the IR region. In some embodiments, the light source produces light with a wavelength of 300 nm, 350 nm, 400 nm, 450 nm, 500 nm, 550 nm, 600 nm, 650 nm, 700 nm, 750 nm, 800 nm, 850 nm, 900 nm, 950 nm, and 1000 nm, or a range bounded by any two of the aforementioned numbers, and/or about any of the aforementioned numbers. In some embodiments, the light source produces light with a wavelength below about 600 nm, below about 575 nm, below about 550 nm, below about 540 nm, below about 530 nm, below about 520, or below a range bounded by any two of the aforementioned numbers, and/or below about any of the aforementioned numbers. In some embodiments, the light source is a laser. In other embodiments, the light source is a lamp combined with a monochromator. In some embodiments, the light source is substantially monochromatic. In some embodiments, the light source is part of a Raman spectrometer.

### Markers

Suitable markers for use in embodiments include compounds that produce a detectable change as a function of metabolic activity. In some embodiments, suitable markers are chemical compounds. Examples of chemical compounds include, but are not limited to anti-oxidants, free radical scavengers, carotenoids (including the classes of xanthophylls and carotenes), organic pigments, and dyes. Specific examples of chemical compound markers include lycopene and beta-carotene. Additional markers include those that transmit light between about 1150 cm⁻¹ and about 1165 cm⁻¹, as well as markers that transmit light between about 1500 cm⁻¹ and about 1550 cm⁻¹. In some embodiments, ROS activating dyes are used as markers.

In other embodiments, and described in more detail above, suitable markers are proteins, including protein complexes. Element-protein complexes are one example of a marker. One specific element-protein complex is an iron-protein complex, and more specifically an Fe-Tr complex.

In some embodiments, markers produce amplified signals. Resonant Raman is one example of an amplified signal. It is understood that resonant Raman is a function of the marker's absorption spectrum (See, i.e., Ermakov et al., Journal of Biomedical Optics, 2005, 10(6): 064028, which is incorporated herein by reference in its entirety). In some embodiments, the light source and its wavelength are selected to produce a resonance enhancement by the marker. For example, beta-carotene has a resonant Raman enhancement at wavelengths below about 525 nm. Similarly, lycopene has a resonance Raman enhancement at about 532 nm. Additionally, the Fe-Tr complex has a resonance Raman enhancement between about 550 nm to about 400 nm.

Several other embodiments that combine various other measurement techniques with a built in amplification method can be constructed using the principles outlined here. As an example, dielectric resonance or relaxation spectroscopy can be used to excite one of an electronic polarization, atomic polarization, dipole relaxation, or ionic relaxation associated with the production, scavenging, or modification of one of the metabolic products or byproducts. Since dielectric resonance processes can amplify the signal associated with the analyte being monitored (for instance, if the frequency of the alternating electromagnetic wave resonates with the specific mode being probed), the resultant analyte signature can be amplified by a factor greater than 1.

Another potential set of embodiment would include a probe that measures a resonant process, combined with another probe that measures another process (the 2^{nd} process could be resonant or non-resonant) in a manner that improves the diagnostic efficiency. As an example, an embodiment could combine resonant Raman methods (similar to what we have outlined) with an alternating electromagnetic field of a frequency that resonates with one of the dielectric modes. This combination could be implemented with a 2 dimensional correlation methodology, that increases the signal to noise ratio by another factor of 10, compared to resonant Raman methods alone. Another potential embodiment exploits surface enhanced Raman spectroscopy (SERS) methods to detect free radical production during microbial cell metabolism. According to the disclosed methods, a surface that results in SERS amplification (such as gold nanoparticles coated on a glass surface) would be combined with a surface chemistry that preferentially attracts all microbial cells (an example of this would be a lipid layer) and a free radical scavenger (such as the lycopene lipoprotein complex). The SERS amplification would enhance the signature associated with lycopene and all other components adsorbed onto the lipid surface, compared to all serum components that are not adsorbed onto the surface. Thus, any changes to the free radical scavengers adsorbed onto the surface, or any production/modification of any lipid friendly metabolic byproducts could be detected.

In some embodiments, the marker is naturally produced in the sample. In other embodiments, the marker is added to the sample. In some embodiments, the marker is naturally occurring but additional marker is added to the sample to increase its concentration. The addition of any marker is optionally before, during, or after one or more data collections. In some embodiments, the marker is added prior to illuminating the sample with the illuminating/light source.

Additionally, in some embodiments a marker is added to the surface of the sample container to afford a method for calibrating the system or signals (either via the presence of the calibrating marker or the intensity of a signal from the calibrating marker). In other embodiments, the calibrating marker is incorporated or doped into the sample container material. In some embodiments, the sample container is coated with a calibrating marker that is metal oxide. In some embodiments, the metal oxide calibrating marker is Vanadium oxide. In other embodiments, the metal oxide calibrating marker is Aluminum oxide. The metal oxide can be applied via known methods or incorporated via known methods. One such application method is a sputter coat technique. Sputter coating is especially adapted for applying metal oxides when the sample container is made of glass. However, the sample container can be made of other materials, such as a plastic that does not interfere with the Raman spectra from the analyte being monitored. In some embodiments the sample container is itself a calibrating marker. The intensity of the signal, such as a Raman signal, produced by the metal oxide is a function of the thickness of the coating on the sample container. The thickness of the coating on the container can be controlled by the sputtering process. In some embodiments, the thickness is 0.5 nm, 1 nm, 1.5 nm, 2 nm, 2.5 nm, 3 nm, 3.5 nm, 4 nm, 4.5 nm, 5 nm, 5.5 nm, 6 nm, 6.5 nm, 7 nm, 7.5 nm, 8 nm, 8.5 nm, 9 nm, 9.5 nm, 10 nm, 11 nm, 13 nm, 15 nm, 20 nm, 50 nm, and 100 nm, or a range bounded by any two of the aforementioned numbers, and/or about any of the aforementioned numbers.

In some embodiments a calibrating marker is added to the sample. The calibrating marker can be a metal oxide or a nanoparticle. In some embodiments, the calibrating marker is a metal oxide nanoparticle. Examples of metal oxides include Titanium dioxide. Examples of a metal oxide nanoparticle includes anatase titania. The anatase titania form of titanium dioxide exhibits a Raman signal at 640 cm⁻¹. The desired intensity of the calibrating marker is a function of concentration of the calibrating marker in the sample. Metal oxides, nanoparticles, and metal oxide nanoparticles are also suitable for doping or incorporating into the sample container material.

### Metabolic Activity

As described above, metabolic activity in a sample is detected by a change in a signal from the marker. In some embodiments, metabolic activity is detected by changes in the light transmitted by the marker. Such changes include, but are not limited to, changes in intensity of one or more wavelengths of the transmitted light. The presence of such changes indicates metabolic activity in the sample. The absence of such changes indicates a lack of metabolic activity in the sample. Metabolic activity indicates the presence of a substance in the sample, such as a pathogen. In some embodiments, the detected metabolic activity is attributed to the pathogen. In other embodiments, the metabolic activity is attributed to a non-pathogenic constituent in the sample, such as a cell. In some embodiments, intensity of the transmitted light increases when metabolic activity occurs. In other embodiments, intensity of the transmitted light decreases when metabolic activity occurs.

In some embodiments, the metabolic activity is the sequestering of nutrients that are needed for a pathogen's metabolism. In some embodiments, the nutrient is an element, such as iron, and the sequestering is performed by an enzyme, such as transferrin.

In other embodiments, the metabolic activity is the production of free radicals. In some embodiments, the free radicals are reactive oxygen species (“ROS”). In other embodiments, the free radicals are reaction nitrogen species (“RNS”). In some embodiments, the marker scavenges free radicals and is depleted.

In some embodiments, the metabolic activity is the production of antioxidants. Consequently, in some embodiments the metabolic activity results in the production of a marker, and the concentration of the marker increases over time. Moreover, some anti-oxidants are free radical scavengers. Thus some embodiments have the production of free radical scavengers as detectable metabolic activity. In other embodiments, the metabolic activity is the production of one or more carotenoids.

As a representative procedure for detecting metabolic activity and making decisions based thereon, some embodiments measure a “slope” which is the time derivative of a marker associated with microbial activity. This estimate of slope optionally includes a confidence interval around it, and in some embodiments, a diagnostic decision is made when the slope +/− confidence interval is within an “uninfected” or “infected” band. In some embodiments, the positions of the infected/uninfected bands are set via a calibration with known samples that have been artificially inoculated with known levels of bacteria. In some embodiments, the positions of the infected/uninfected bands are confirmed by clinical studies with patient samples.

Additional embodiments use other algorithms for estimating metrics that correspond to the slope and confidence interval. These algorithms include, but are not limited to Eigen Value decomposition and principal component analysis. With this algorithm, the first few principle components (e.g, the first 3) can be taken as a measure of the signal and all higher components can be used surrogate measures of the confidence interval or noise.

### Pathogens

In some embodiments, the detection of metabolic activity is indicative of the presence of a foreign substance in the sample. These foreign substances include, but are not limited to pathogens. In some embodiments, the pathogen is a bacterium. Non-limiting examples of specific bacteria include the following species: S. aureus, A. baumannii, K. pneumoniae, and Escherichia coli. In some embodiments, the pathogen is a fungus or mould. Non-limiting examples of specific fungi include the following: Candida albicans. In some embodiments, the pathogen is a parasite. Moreover, in some embodiments, the pathogen is a virus. In some embodiments, two or more types of pathogens are present.

### Time Points

In one embodiment, the time points at which a sample is illuminated are not particularly limited. In some embodiments, the time points are between zero and four weeks, zero and three weeks, zero and two weeks, and/or zero and one week, and/or about any of the aforementioned numbers. In other embodiments, the time points are between zero and 7 days, zero and 6 days, zero and 5 days, zero and 4 days, zero and 3 days, zero and 2 days, and/or zero and 1 day, and/or about any of the aforementioned numbers. In some embodiments, the time points are between zero and 24 hours, zero and 23 hours, zero and 22 hours, zero and 21 hours, zero and 20 hours, zero and 19 hours, zero and 18 hours, zero and 17 hours, zero and 16 hours, zero and 15 hours, zero and 14 hours, zero and 13 hours, zero and 12 hours, zero and 11 hours, zero and 10 hours, zero and 9 hours, zero and 8 hours, zero and 7 hours, zero and 6 hours, zero and 5 hours, zero and 4 hours, zero and 3 hours, zero and 2 hours, and/or zero and 1 hour, and/or about any of the aforementioned numbers. In other embodiments, the time points are between zero and 120 minutes, zero and 110 minutes, zero and 100 minutes, zero and 90 minutes, zero and 80 minutes, zero and 70 minutes, 60 minutes, zero and 50 minutes, zero and 40 minutes, zero and 30 minutes, zero and 20 minutes, zero and 10 minutes, zero and 5 minutes, zero and 4 minutes, zero and 3 minutes, zero and 2 minutes, and/or zero and 1 minute, and/or about any of the aforementioned numbers.

In one embodiment, the method for detecting metabolic activity is completed in less than four weeks, three weeks, two weeks, one week, or a range bounded by any two of the aforementioned numbers, and/or about any of the aforementioned numbers. In some embodiments, the method for detecting metabolic activity is completed in less than 7 days, 6 days, 5 days, 4 days, 3 days, 2 days, 1 day, or a range bounded by any two of the aforementioned numbers, and/or about any of the aforementioned numbers. In other embodiments, the method for detecting metabolic activity is completed in less than 24 hours, 23 hours, 22 hours, 21 hours, 20 hours, 19 hours, 18 hours, 17 hours, 16 hours, 15 hours, 14 hours, 13 hours, 12 hours, 11 hours, 10 hours, 9 hours, 8 hours, 7 hours, 6 hours, 5 hours, 4 hours, 3 hours, 2 hours, 1 hour, or a range bounded by any two of the aforementioned numbers, and/or about any of the aforementioned numbers. In some embodiments, the method for detecting metabolic activity is completed in less than 120 minutes, 110 minutes, 100 minutes, 90 minutes, 80 minutes, 70 minutes, 60 minutes, 50 minutes, 40 minutes, 30 minutes, 20 minutes, 10 minutes, 5 minutes, 4 minutes, 3 minutes, 2 minutes, 1 minute, or a range bounded by any two of the aforementioned numbers, and/or about any of the aforementioned numbers.

In one embodiment, the number of time points at which a sample is illuminated is not particularly limited. In some embodiments, the number of time points at which a sample is irradiated is 1 time, 2 times, 3 times, 4 times, 5 times, 6 times, 7 times, 8 times, 9 times, 10 times, 15 times, 20 times, 25 times, 30 times, 40 times, 50 times, 60 times, 70 times, 80 times, 90 times, 100 times, or a range bounded by any two of the aforementioned numbers, and/or about any of the aforementioned numbers. In other embodiments, the number of time points at which a sample is illuminated is at least 1 time, at least 2 times, at least 3 times, at least 4 times, at least 5 times, at least 6 times, at least 7 times, at least 8 times, at least 9 times, at least 10 times, at least 15 times, at least 20 times, at least 25 times, at least 30 times, at least 40 times, at least 50 times, at least 60 times, at least 70 times, at least 80 times, at least 90 times, and/or at least 100 times, and/or about any of the aforementioned numbers. In some embodiments, the number of time points at which a sample is illuminated is less than 2 times, less than 3 times, less than 4 times, less than 5 times, less than 6 times, less than 7 times, less than 8 times, less than 9 times, less than 10 times, less than 15 times, less than 20 times, less than 25 times, less than 30 times, less than 40 times, less than 50 times, less than 60 times, less than 70 times, less than 80 times, less than 90 times, and/or less than 100 times, and/or about any of the aforementioned numbers.

It is understood that a plurality of time points may be utilized in the described embodiments. In such instances, a plurality of time points includes continuous measurement and competitive measurement at discrete time points.

### EXAMPLES

Pathogen Detection with Lycopene Marker

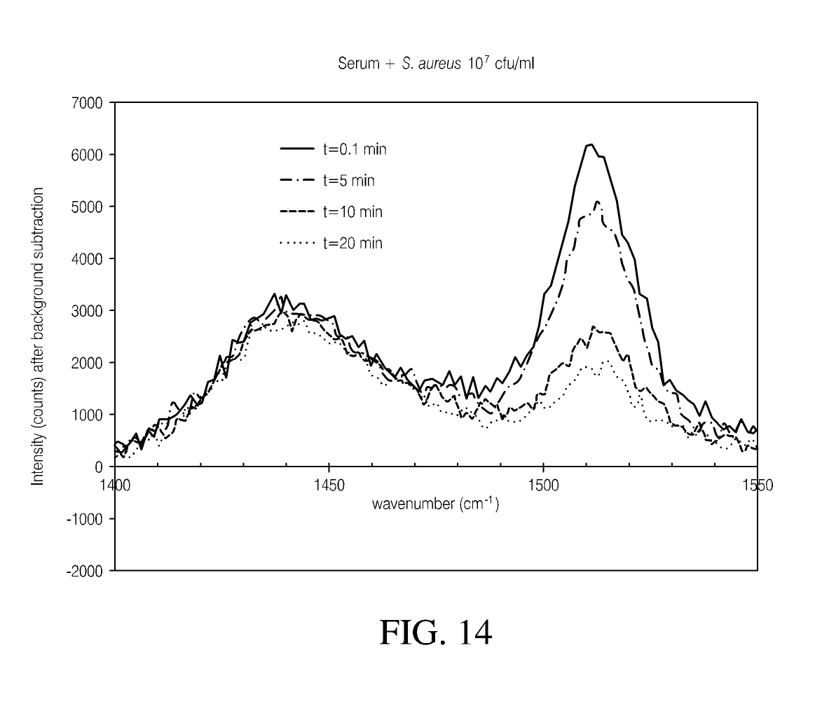

A serum sample was inoculated with 10⁷ cfu/mL methicillin-resistant Staphylococcus aureus (“MRSA”). Using a 532 nm wavelength, the Raman scattering profiles were measured as a function of time. The peak at 1516 cm⁻¹ was attributed to lycopene, and it was observed that this peak decreased over time reflecting the consumption of lycopene by metabolically active bacteria, as indicated by the 4 traces corresponding to 0.1 minutes, 10 minutes, 15 minutes and 20 minutes (See FIG. 14). Although not depicted in FIG. 14, the other lycopene peaks at 1156 cm⁻¹ also show a similar behavior.

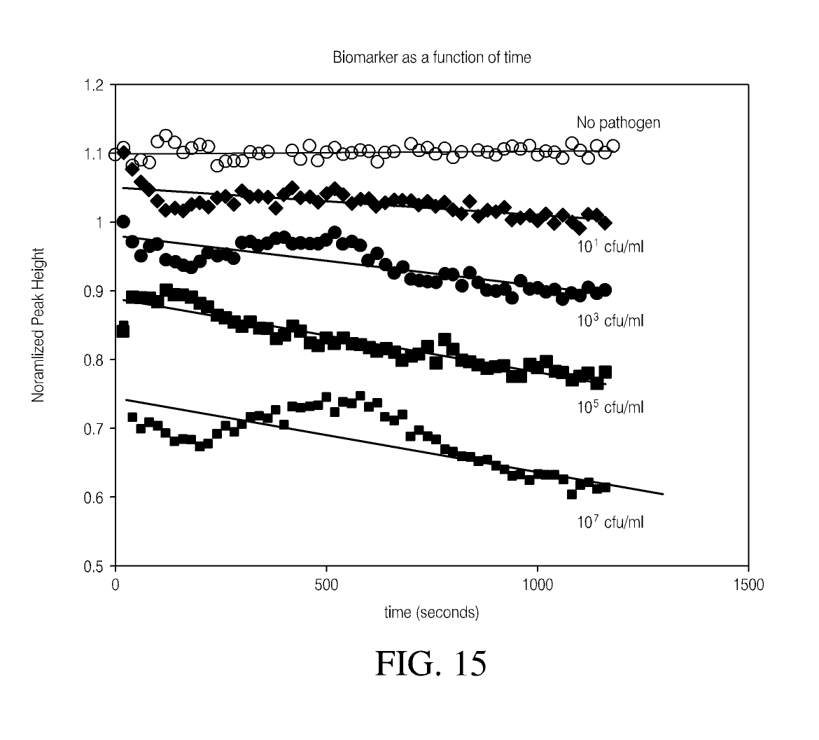

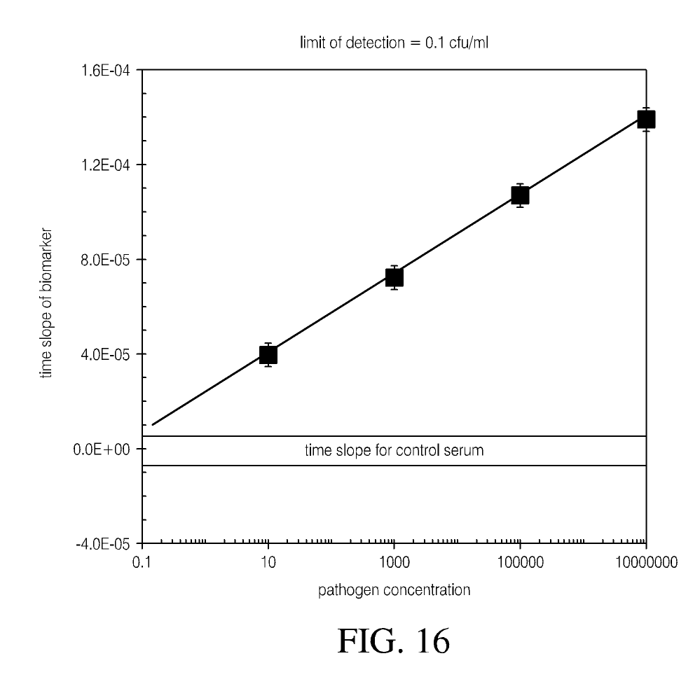

FIG. 15 illustrates the normalized intensity v. time profile of the 1516 cm⁻¹ lycopene peak in the serum of a healthy volunteer, with 4 parts added broth and various amounts (no pathogen, 10¹ cfu/mL, 10³ cfu/mL, 10⁵ cfu/mL, and 10⁷ cfu/mL) of added S. aureus bacteria. In all cases, for samples with added bacteria, the peak heights decrease as a function of time. By contrast, the height of the control sample (with no added pathogen) remains nearly independent of time. Further, as the amount of added bacteria increases, the intensity of the lycopene peak decreases at a faster rate. FIG. 16 illustrates the slope (i.e., of the time derivative) of all the profiles depicted in FIG. 15, as a function of added pathogen concentration. Also depicted is a band that depicts the slope of the control sample. In all cases, the width of the band and the uncertainty correspond to the 95% Confidence Interval (95CI) of the estimated slope.

Antimicrobial Susceptibility and/or Pathogen Identification via Antimicrobial Susceptibility

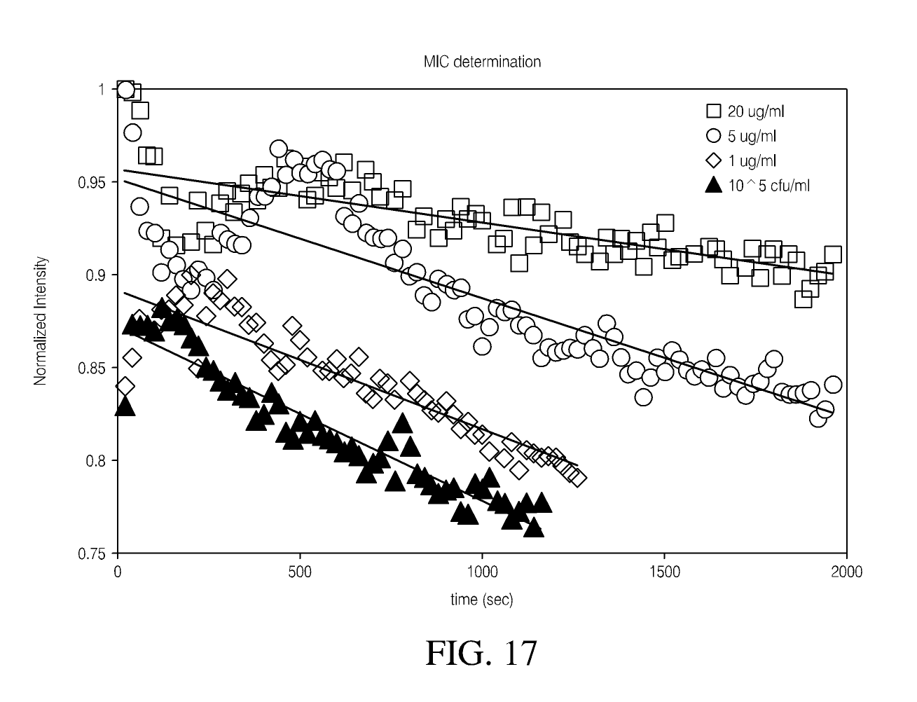

FIGS. 17 & 18 illustrate the method by which a marker can be used to estimate the minimum inhibitory concentration MIC. FIG. 17 illustrates the Raman peak height at 1516 cm⁻¹ (which has been ascribed to lycopene) as a function of time for several samples. The samples include a serum sample that has been artificially inoculated with S. aureus to a concentration of 10⁵ cfu/mL, and additional samples that also contain various concentrations of vancomycin (1, 5 and 20 μg/mL) as indicated in the legend. As depicted in the figure, the peak intensity decreases in all cases, but it decreases at the fastest rate for the sample that does not have any added vancomycin, and the slope progressively decreases as more vancomycin is added to the assay.

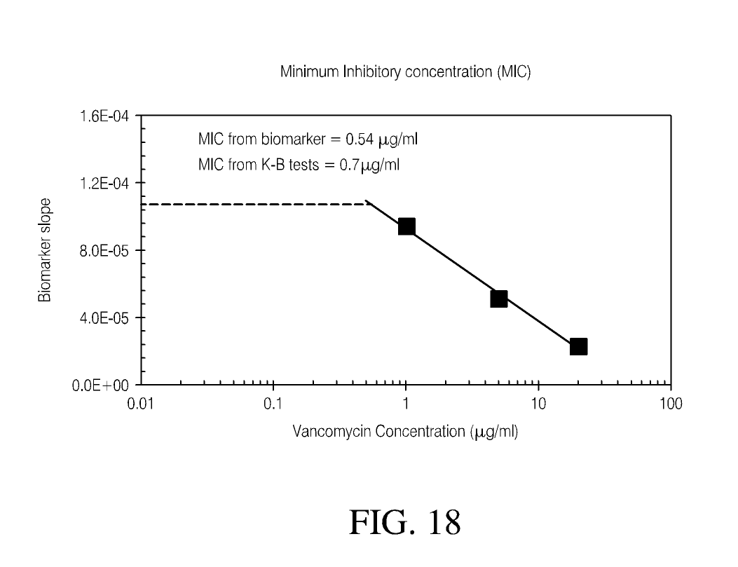

FIG. 18 plots the slope of all the traces in FIG. 17, as a function of added vancomycin concentration. The dashed horizontal line corresponds to the slope of the sample without any added vancomycin, and the 3 data points correspond to vancomycin concentrations of 1, 5 and 20 μg/mL. The solid black line is a logarithmic fit of the 3 data points, and the MIC corresponds to the point at which the solid black line intersects the dashed line. In this particular example, the estimated MIC is 0.54 μg/mL, which is very close to the MIC that is estimated from traditional Kirby Bauer tests (0.7 μg/mL).

FIGS. 19 and 20 depict the process by which drug efficacy is characterized against a sample that is co-infected with more than one organism. FIG. 19 depicts the peak height at 1516 cm⁻¹ (which is ascribed to lycopene) as a function of time. In this example, the sample was co-infected with both Gram positive and Gram negative bacteria. The 4 traces depict the lycopene peak height as a function of time for the sample with added vancomycin (20 μg/mL), added ceftazidime (20 μg/mL) and with both vancomycin and ceftazidime (both at 20 μg/mL). Since Gram negative bacteria are generally resistant to vancomycin, and gram positive bacteria are generally resistant to ceftazidime, we expect both drugs to have partial efficacy when used in isolation. By contrast, when the two drugs are used in combination, then we expect to have maximum efficacy. This is consistent with the observation˜the time slope of the lycopene peak height is smallest for the sample with added vancomycin and ceftazidime. FIG. 20 depicts the slopes of these traces as a function of drug concentration, so as to depict the drug effectiveness in a manner similar to that in FIG. 18.

### Detecting Metabolic Activity via Marker Production

The presence of pathogens can also manifest as the production of various markers that are resonantly amplified. One specific example is the production of various carotenoids by certain microorganisms. The ability of some microorganisms to synthesize carotenoids in light is one of the most delicate inventions of nature aimed at protecting the cells from harmful effects of UV light exposure and or other sources of reactive oxygen species. Photoinduced carotenogenesis has been documented in several bacteria, such as mycobacterial species (Kolmanová A, Hochmannová J, Malek I. Carotenoids synthesized by UV-induced mutants of a non-acid-fast strain of Mycobacterium phlei. Folia Microbiol. (Praha) 1970; 15(6):426-430; Houssaini-Iraqui M, Lazraq M H, Clavel-Sérès S, Rastogi N, David H L. Cloning and expression of Mycobacterium aurum carotenogenesis genes in Mycobacterium smegmatis. FEMS Microbiol. Lett. 1992 January; 69(3):239-244). It is likely that accumulation of carotenoids is necessitated by the need for survival against an oxidative burst.

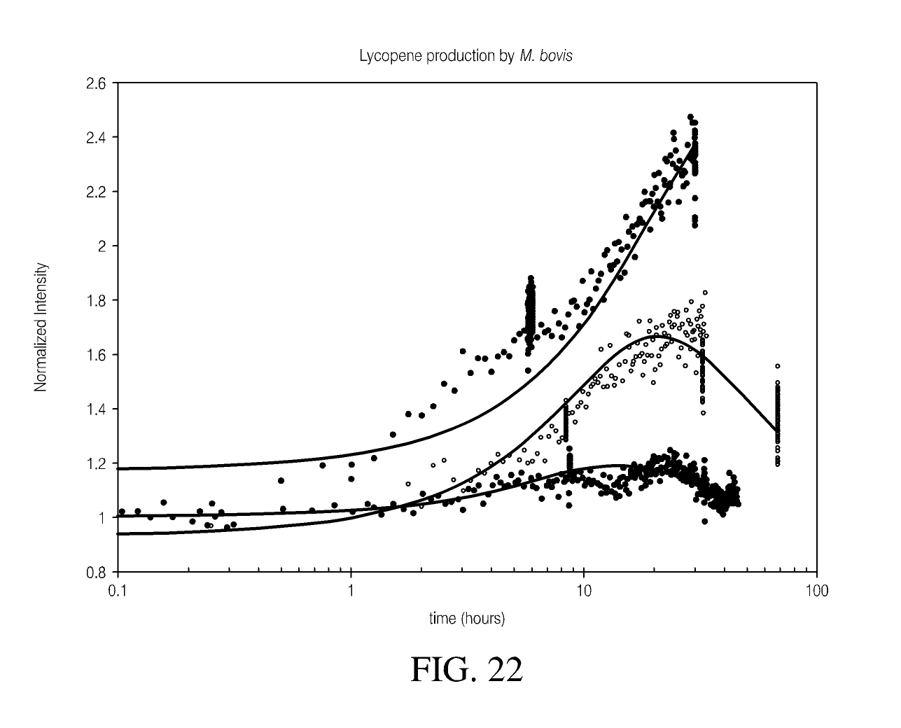

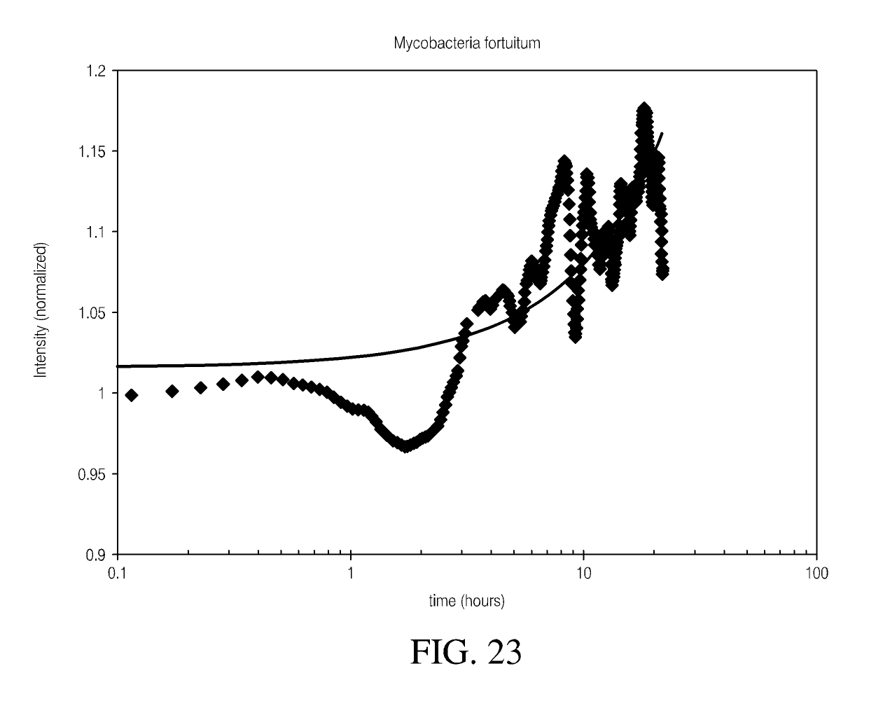

FIG. 21 depicts the production of lycopene in a sample that contains Mycobacterium bovis (M. bovis) to a concentration of 10 cfu/mL. This increase is manifest in the intensity of the 1516 cm⁻¹ Raman peak, which is ascribed to lycopene. A control sample that does not contain any added M. bovis does not produce lycopene. Since lycopene can also be consumed by pathogen metabolism, different conditions can result in an initial increase in the lycopene intensity followed by a subsequent decrease, as depicted in FIG. 22. FIG. 23 depicts a similar lycopene production in a sample containing M. fortiutum.

### Detection of Toxins

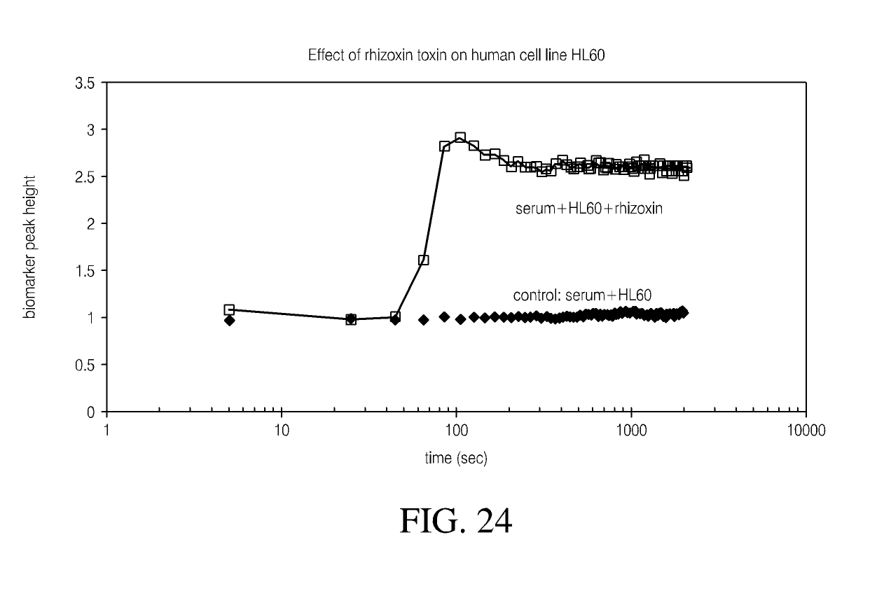

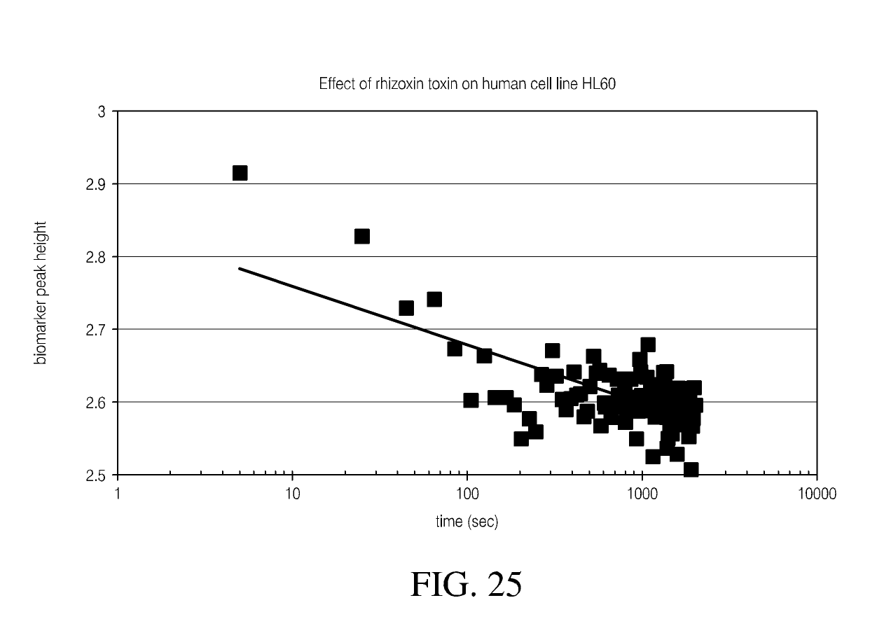

FIG. 24 depicts the results of one toxin detection experiment. A control sample comprising human serum (centrifuged from the blood collected from a healthy volunteer) was combined with trypticase soy broth and HL60 human cells. As expected, the control sample did not demonstrate any change in the lycopene peak height as measured by Raman spectroscopy. This result is consistent with negligible free radical production by the human cell line when it is undergoing normal metabolism. By contrast, when 4 μM of the toxin rhizoxin is added to the assay, then the lycopene peak increases significantly at first, followed by a steady decline consistent with stressed metabolism of the human cell line. FIG. 25 plots the normalized intensity for the 1516 cm⁻¹ peak as a function of time for the rhizoxin containing sample. The plot clearly shows a decrease in the intensity of the normalized signal over time.

### REFERENCES

Unless otherwise specified, all references cited herein are incorporated by reference in their entirety.

### CONCLUSION

While the invention has been described with reference to the specific embodiments thereof, it should be understood by those skilled in the art that various changes may be made and equivalents may be substituted without departing from the true spirit and scope of the invention. This includes embodiments which do not provide all of the benefits and features set forth herein. In addition, many modifications may be made to adapt a particular situation, material, composition of matter, process, process step or steps, to the objective, spirit and scope of the described embodiments. All such modifications are intended to be within the scope of the claims appended hereto. Accordingly, the scope of the invention is defined only by reference to the appended claims.

What is claimed is:

1. A method for detecting metabolic activity in a sample, comprising:
obtaining a sample;
illuminating the sample with substantially monochromatic light at a plurality of time points;
measuring Raman scattered light from a chemical marker of metabolic activity in the sample at the plurality of time points; and
detecting metabolic activity from a change in the Raman scattered light at the plurality of time points.

2. A method for detecting metabolic activity in a sample, comprising:
providing a sample having a detectable marker therein that is reflective of metabolic activity in the sample;
producing an amplified signal from the marker;
measuring the amplified signal at a plurality of time points; and
detecting metabolic activity from a change in the amplified signal at the plurality of time points.

3. The method of any one of claims 1-2, wherein the Raman scattered light is resonance enhanced.

4. The method of any one of claims 1-3, wherein the marker is an anti-oxidant.

5. The method of any one of claims 1-4, wherein the marker is a free-radical scavenger.

6. The method of any one of claims 1-5, wherein the marker is a carotenoid.

7. The method of any one of claims 1-6, wherein the marker is lycopene.

8. The method of any one of claims 1-3, wherein the marker is an element-sequestering protein complex.

9. The method of any one of claims 1-3 and 8, wherein the marker is an iron-sequestering protein complex.

10. The method of any one of claims 1-7, wherein the metabolic activity is the production of free radicals.

11. The method of any one of claims 1-7, wherein the metabolic activity is the production of a carotenoid.

12. The method of any one of claims 1-3 and 8-9, wherein the metabolic activity is the sequestering of iron.

13. The method of any one of claims 1-12, wherein the change in the Raman scattered light is cumulative.

14. The method of any one of claims 1-13, wherein the presence of metabolic activity indicates the presence of a pathogen.

15. The method of any one of claims 1-14, wherein the presence of metabolic activity indicates the presence of a bacterium, fungus, parasite, or virus.

16. The method of any one of claims 1-15, wherein the amount of the marker increases as a result of the metabolic activity.

17. The method of any one of claims 1-15, wherein the amount of the marker decreases as a result of the metabolic activity.

18. The method of any one of claims 1-17, wherein the sample contains an anti-pathogenic substance.

19. The method of any one of claims 1-18, wherein the sample contains an anti-pathogenic substance configured to allow pathogen classification from the detected metabolic activity.

20. The method of any one of claims 1-18, wherein the sample contains a culture broth configured to allow pathogen classification from the presence of metabolic activity.

21. The method of any one of claims 1-20, wherein the sample contains an anti-pathogenic substance selected from the group consisting of an anti-biotic, anti-fungal, and anti-viral substance; and wherein the detected metabolic activity indicates effectiveness or ineffectiveness of the anti-pathogenic substance.

22. The method of any one of claims 1-21, wherein the sample includes a body fluid.

23. The method of any one of claims 1-22, wherein the sample includes a body fluid selected from the group consisting of blood, cerebrospinal fluid, and urine.

24. The method of any one of claims 1-23, wherein the sample is cultured.

25. The method of any one of claims 1-24, wherein the sample includes a cultured cell line.

26. The method of any one of claims 1-25, wherein the marker is naturally present in the sample.

27. The method of any one of claims 1-26, wherein the marker is added to the sample prior to illuminating.

28. The method of any one of claims 1-27, wherein the detecting is completed in less than about 6 hours.

29. The method of any one of claims 1-28, wherein the detecting is completed in less than about 30 minutes.

30. The method of any one of claims 1-29, wherein the sample includes a calibrant Raman marker in the sample or on a sample container.

31. The method of claim 30, further comprising: interrogating the sample or the sample container for the presence or intensity of the calibrant Raman marker.

32. The method of any one of claims 1-7, 10, 13, 17-18, and 22-31, wherein the metabolic activity indicates that a toxic substance is present in the sample.

33. A system for detecting metabolic activity in a sample, comprising:
a light source;
a controller for periodically illuminating a sample with the light source, wherein the sample contains a chemical marker responsive to metabolic activity in the sample;
a detector configured to measure a light signal from the marker; and
a computer configured to receive light measurements from the detector and ascertain a change in the marker over time that is indicative of metabolic activity in the sample.

34. The system of claim 33, wherein the marker is an anti-oxidant or iron-sequestering protein complex.

35. The system of any one of claims 33-34, wherein the marker is a free-radical scavenger.

36. The system of any one of claims 33-35, wherein the marker is a carotenoid.

37. The system of any one of claims 33-36, wherein the marker is lycopene.

38. The system of any one of claims 33-37, wherein the marker is non-naturally occurring in the sample.

39. The system of any one of claims 33-37, wherein the marker is naturally occurring in the sample.

40. The system of any one of claims 33-39, wherein the light signal from the marker is from Stokes Raman scattering.

41. The system of any one of claims 33-39, wherein the light signal from the marker is from anti-Stokes Raman scattering.

42. The system of any one of claims 33-41, wherein the amount of the marker accumulates in the sample over time to indicate metabolic activity in the sample.

43. The system of any one of claims 33-41, wherein the marker in the sample decreases over time to indicate metabolic activity in the sample.

44. The system of any one of claims 33-43, wherein the light transmitted by the marker is resonance enhanced.

45. The system of any one of claims 33-44, wherein the metabolic activity is the production of free radicals.

46. The system of any one of claims 33-44, wherein the metabolic activity is the sequestering of iron.

47. The system of any one of claims 33-46, wherein the light transmitted by the marker is cumulative.

48. The system of any one of claims 33-47, wherein the presence of metabolic activity indicates the presence of a pathogen.

49. The system of any one of claims 33-48, wherein the presence of metabolic activity indicates the presence of a bacterium, fungus, parasite, or virus.

50. The system of any one of claims 33-49, wherein the sample contains an anti-pathogenic substance.

51. The system of any one of claims 33-50, wherein the sample contains an anti-pathogenic substance configured to allow pathogen classification from the presence of metabolic activity.

52. The system of any one of claims 33-51, wherein the sample contains a culture broth configured to allow pathogen classification from the presence of metabolic activity.

53. The system of any one of claims 33-52, wherein the sample contains an anti-pathogenic substance selected from the group consisting of an anti-biotic, anti-fungal, and anti-viral substance; and wherein the detected metabolic activity indicates effectiveness or ineffectiveness of the anti-pathogenic substance.

54. The system of any one of claims 33-53, wherein the sample includes a body fluid.

55. The method of any one of claims 33-54, wherein the time is less than about 6 hours.

56. The method of any one of claims 33-55, wherein the time is less than about 30 minutes.

57. The method of any one of claims 33-56, wherein the signal is resonance enhanced Raman light scattering.

58. The method of any one of claims 33-41, 43-45, 47, 50, and 54-57, wherein the metabolic activity indicates that a toxic substance is present in the sample.

59. The system of any one of claims 33-58, wherein the sample includes a calibrant Raman marker in the sample or on a sample container.

60. The system of claim 59, wherein the detector is configured to measure the sample or the sample container for the presence or intensity of the calibrant Raman marker.
